SEQUENCE LISTING (37 sequences)

SEQ ID NO: 1 - protein, 93 aa, Artificial Sequence
  fibronectin type III (FN3) scaffold, Nsa1
SSVPTKLEVVAATPTSLLISWDAPAVTVDYYVITYGETGSGGYAWQEFEVPGSKSTATISGLKPGVDYTITVYAGYYGYPTYYSSPISINYRT

SEQ ID NO: 2 - protein, 89 aa, Artificial Sequence
  FN3 scaffold, Nsa5
SSVPTKLEVVAATPTSLLISWDAPAVTVDFYVITYGETGYFSGFQEFEVPGSKSTATISGLKPGVDYTITVYAGYYGYYGSPISINYRT

SEQ ID NO: 3 - protein, 6 aa, Artificial Sequence
  CD loop of FN3 scaffold, Nsa1
SGGYAW

SEQ ID NO: 4 - protein, 12 aa, Artificial Sequence
  FG loop of FN3 scaffold, Nsa1
AGYYGYPTYYSS

SEQ ID NO: 5 - protein, 5 aa, Artificial Sequence
  CD loop of FN3 scaffold, Nsa5
YFSGF

SEQ ID NO: 6 - protein, 9 aa, Artificial Sequence
  FG loop of FN3 scaffold, Nsa5
AGYYGYYGS

SEQ ID NO: 7 - protein, 335 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 8 - protein, 145 aa, Homo sapiens
KRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 9 - protein, 208 aa, Homo sapiens
MDPFLVLLHSVSSSLSSSELTELKFLCLGRVGKRKLERVQSGLDLFSMLLEQNDLEPGHTELLRELLASLRRHDLLRRVDDFEAGAAAGAAPGEEDLCAAFNVICDNVGKDWRRLARQLKVSDTKIDSIEDRYPRNLTERVRESLRIWKNTEKENATVAHLVGALRSCQMNLVADLVQEVQQARDLQNRSGAMSPMSWNSDASTSEAS

SEQ ID NO: 10 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 11 - protein, 311 aa, Homo sapiens
MAAGQNGHEEWVGSAYLFVESSLDKVVLSDAYAHPQQKVAVYRALQAALAESGGSPDVLQMLKIHRSDPQLIVQLRFCGRQPCGRFLRAYREGALRAALQRSLAAALAQHSVPLQLELRAGAERLDALLADEERCLSCILAQQPDRLRDEELAELEDALRNLKCGSGARGGDGEVASAPLQPPVPSLSEVKPPPPPPPAQTFLFQGQPVVNRPLSLKDQQTFARSVGLKWRKVGRSLQRGCRALRDPALDSLAYEYEREGLYEQAFQLLRRFVQAEGRRATLQRLVEALENELTSLAEDLLGLTDPNGGLA

SEQ ID NO: 12 - protein, 552 aa, Homo sapiens
MDNMSITNTPTSNDACLSIVHSLMCHRQGGESETFAKRAIESLVKKLKEKKDELDSLITAITTNGAHPSKCVTIQRTLDGRLQVAGRKGFPHVIYARLWRWPDLHKNELKHVKYCQYAFDLKCDSVCVNPYHYERVVSPGIDLSGLTLQSNAPSSMMVKDEYVHDFEGQPSLSTEGHSIQTIQHPPSNRASTETYSTPALLAPSESNATSTANFPNIPVASTSQPASILGGSHSEGLLQIASGPQPGQQQNGFTGQPATYHHNSTTTWTGSRTAPYTPNLPHHQNGHLQHHPPMPPHPGHYWPVHNELAFQPPISNHPAPEYWCSIAYFEMDVQVGETFKVPSSCPIVTVDGYVDPSGGDRFCLGQLSNVHRTEAIERARLHIGKGVQLECKGEGDVWVRCLSDHAVFVQSYYLDREAGRAPGDAVHKIYPSAYIKVFDLRQCHRQMQQQAATAQAAAAAQAAAVAGNIPGPGSVGGIAPAISLSAAAGIGVDDLRRLCILRMSFVKGWGPDYPRQSIKETPCWIEIHLHRALQLLDEVLHTMPIADPQPLD

SEQ ID NO: 13 - protein, 230 aa, Homo sapiens
WCSIAYFEMDVQVGETFKVPSSCPIVTVDGYVDPSGGDRFCLGQLSNVHRTEAIERARLHIGKGVQLECKGEGDVWVRCLSDHAVFVQSYYLDREAGRAPGDAVHKIYPSAYIKVFDLRQCHRQMQQQAATAQAAAAAQAAAVAGNIPGPGSVGGIAPAISLSAAAGIGVDDLRRLCILRMSFVKGWGPDYPRQSIKETPCWIEIHLHRALQLLDEVLHTMPIADPQPLD

SEQ ID NO: 14 - protein, 316 aa, Homo sapiens
MFQAAERPQEWAMEGPRDGLKKERLLDDRHDSGLDSMKDEEYEQMVKELQEIRLEPQEVPRGSEPWKQQLTEDGDSFLHLAIIHEEKALTMEVIRQVKGDLAFLNFQNNLQQTPLHLAVITNQPEIAEALGAGCDPELRDFRGNTPLHLACEQGCLASVGVLTQSCTTPHLHSILKATNYNGHTCLHLASIHGYLGIVELLVSLGADVNAQEPCNGRTALHLAVDLQNPDLVSLLLKCGADVNRVTYQGYSPYQLTWGRPSTRIQQQLGQLTLENLQMLPESEDEESYDTESEFTEFTEDELPYDDCVFGGQRLTL

SEQ ID NO: 15 - protein, 729 aa, Artificial Sequence
  CD45 trans-membrane and endodomain sequence
ALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 16 - protein, 362 aa, Artificial Sequence
  CD148 trans-membrane and endodomain sequence
AVFGCIFGALVIVTVGGFIFWRKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIA

SEQ ID NO: 17 - protein, 595 aa, Artificial Sequence
  Protein tyrosine phosphatase (PTP) PTPN6 sequence
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 18 - protein, 272 aa, Artificial Sequence
  phosphatase domain of PTPN6
FWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 19 - protein, 597 aa, Artificial Sequence
  PTPN11 sequence
MTSRRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQALLQGNTERTVWQYHFRTWPDHGVPSDPGGVLDFLEEVHHKQESIMDAGPVVVHCSAGIGRTGTFIVIDILIDIIREKGVDCDIDVPKTIQMVRSQRSGMVQTEAQYRFIYMAVQHYIETLQRRIEEEQKSKRKGHEYTNIKYSLADQTSGDQSPLPPCTPTPPCAEMREDSARVYENVGLMQQQKSFR

SEQ ID NO: 20 - protein, 351 aa, Artificial Sequence
  phosphatase domain of PTPN11
FWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQALLQGNTERTVWQYHFRTWPDHGVPSDPGGVLDFLEEVHHKQESIMDAGPVVVHCSAGIGRTGTFIVIDILIDIIREKGVDCDIDVPKTIQMVRSQRSGMVQTEAQYRFIYMAVQHYIETLQRRIEEEQKSKRKGHEYTNIKYSLADQTSGDQSPLPPCTPTPPCAEMREDSARVYENVGLMQQQKSFR

SEQ ID NO: 21 - protein, 173 aa, Artificial Sequence
  E3 ubiquitin-protein ligase CHIP degradation motif
RLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWV

SEQ ID NO: 22 - protein, 198 aa, Artificial Sequence
  Simb degradation motif
MMKMETDKIMDETNSNAQAFTTTMLYDPVRKKDSSPTYQTERELCFQYFTQWSESGQVDFVEHLLSRMCHYQHGQINAYLKPMLQRDFITLLPIKGLDHIAENILSYLDAESLKSSELVCKEWLRVISEGMLWKKLIERKVRTDSLWRGLAERRNWMQYLFKPRPGQTQRPHSFHRELFPKIMNDIDSIENNWRTGRH

SEQ ID NO: 23 - protein, 39 aa, Artificial Sequence
  PEST domain sequence
HGFPPEVEEQDDGTLPMSCAQESGMDRHPAACASARINV

SEQ ID NO: 24 - DNA, 30 bp, Artificial Sequence
  Nuclear factor of activated T cells (NFAT) response element
ggaggaaaaactgtttcatacagaaggcgt

SEQ ID NO: 25 - protein, 480 aa, Homo sapiens
MSAEVIHQVEEALDTDEKEMLLFLCRDVAIDVVPPNVRDLLDILRERGKLSVGDLAELLYRVRRFDLLKRILKMDRKAVETHLLRNPHLVSDYRVLMAEIGEDLDKSDVSSLIFLMKDYMGRGKISKEKSFLDLVVELEKLNLVAPDQLDLLEKCLKNIHRIDLKTKIQKYKQSVQGAGTSYRNVLQAAIQKSLKDPSNNFRLHNGRSKEQRLKEQLGAQQEPVKKSIQESEAFLPQSIPEERYKMKSKPLGICLIIDCIGNETELLRDTFTSLGYEVQKFLHLSMHGISQILGQFACMPEHRDYDSFVCVLVSRGGSQSVYGVDQTHSGLPLHHIRRMFMGDSCPYLAGKPKMFFIQNYVVSEGQLEDSSLLEVDGPAMKNVEFKAQKRGLCTVHREADFFWSLCTADMSLLEQSHSSPSLYLQCLSQKLRQERKRPLLDLHIELNGYMYDWNSRVSAKEKYYVWLQHTLRKKLILSYT

SEQ ID NO: 26 - protein, 89 aa, Artificial Sequence
  SHP2-binding FN3-based binding domain, Nsa2
SSVPTKLEVVAATPTSLLISWDAPAVTVDYYVITYGETGYYAYFQEFEVPGSKSTATISGLKPGVDYTITVYAGYYGYYGSPISINYRT

SEQ ID NO: 27 - protein, 89 aa, Artificial Sequence
  SHP2-binding FN3-based binding domain, Nsa4
SSVPTKLEVVAATPTSLLISWDAPAVTVDYYVITYGETGYYAYFQEFEVPGSKSTATISGLKPGVDYTITVYAGYYGYYGNPISINYRT

SEQ ID NO: 28 - protein, 93 aa, Artificial Sequence
  SHP2-binding FN3-based binding domain, Cs1
SSVPTKLEVVAATPTSLLISWDAPAVTVDYYVITYGETGYWPYYWQEFEVPGSKSTATISGLKPGVDYTITVYAGSYDSYYYYGSPISINYRT

SEQ ID NO: 29 - protein, 93 aa, Artificial Sequence
  SHP2-binding FN3-based binding domain, Cs3
SSVPTKLEVVAATPTSLLISWDAPAVTVDYYVITYGETGHWPWVWQEFEVPGSKSTATISGLKPGVDYTITVYAGSYSSYYYYGSPISINYRT

SEQ ID NO: 30 - protein, 48 aa, Artificial Sequence
  XIAP′s RING domain degradation motif
CKICMDRNIAIVFVPCGHLVTCKQCAEAVDKCPMCYTVITFKQKIFMS

SEQ ID NO: 31 - protein, 76 aa, Artificial Sequence
  e4B′s U-box degradation motif
DAPDEFRDPLMDTLMTDPVRLPSGTIMDRSIILRHLLNSPTDPFNRQTLTESMLEPVPELKEQIQAWMREKQNSDH

SEQ ID NO: 32 - protein, 49 aa, Artificial Sequence
  Notch1′s PEST motif degradation motif
HPFLTPSPESPDQWSSSSPHSNVSDWSEGVSSPPTSMQSQIARIPEAFK

SEQ ID NO: 33 - protein, 93 aa, Artificial Sequence
  SHP2-binding FN3-based binding domain, Cs4
SSVPTKLEVVAATPTSLLISWDAPAVTVDYYVITYGETGYWPYYWQEFEVPGSKSTATISGLKPGVDYTITVYAGHYNSYYYSYSPISINYRT

SEQ ID NO: 34 - protein, 4 aa, Artificial Sequence
  sequence motif
KDEL

SEQ ID NO: 35 - protein, 4 aa, Artificial Sequence
  ITAM (immunoreceptor tyrosine-based activation motif)
  misc_feature (2)..(3): Xaa can be any naturally occurring amino acid
  MISC_FEATURE (4)..(4): Xaa may be Leu or Ile
YXXX

SEQ ID NO: 36 - protein, 4 aa, Artificial Sequence
  basic amino acid furin target sequence
  misc_feature (2)..(2): Xaa can be any naturally occurring amino acid
  MISC_FEATURE (3)..(3): Xaa may be Arg or Lys
RXXR

SEQ ID NO: 37 - protein, 7 aa, Artificial Sequence
  consensus Tobacco Etch Virus (TEV) cleavage site
ENLYFQS

(19) United States
(12) Patent Application Publication
(10) Pub. No.: US 2021/0363217 A1
(43) Pub. Date: Nov. 25, 2021

(54) CELL

(71) Applicant: AUTOLUS LIMITED, London (GB)

(72) Inventors: Martin Pulé, London (GB); Shimobi Onuoha, London (GB); Shaun Cordoba, London (GB); Simon Thomas, London (GB); Lenart Senicar, London (GB); Mathieu Ferrari, London (GB)

(21) Appl. No.: 17/253,046

(22) PCT Filed: Jun. 19, 2019
(86) PCT No.: PCT/GB2019/051723

§ 371 Date: Dec. 16, 2020

(30) Foreign Application Priority Data
Jun. 19, 2018 (GB) 1810070.1
Apr. 23, 2019 (GB) 1905652.2

(51) Int. Cl. C07K 14/725; C07K 16/40; A61K 35/17; C07K 16/18

(52) U.S. Cl. CPC C07K 14/7051; C07K 16/40; C07K 2317/569; C07K 16/18; A61K 35/17

(57) ABSTRACT

The present invention provides a cell which expresses: (i) an intracellular single domain protein binder which binds a target protein and modulates an intracellular signalling pathway; and (ii) a chimeric antigen receptor (CAR) or a transgenic T cell receptor (TCR).

### FIELD OF THE INVENTION

The present invention relates to cell which expresses a chimeric antigen receptor (CAR) or a transgenic T cell receptor (TCR). The cell also expresses an intracellular single domain protein binder which binds a target protein and modulates an intracellular signalling pathway.

The single domain protein binder may, for example, modulate apoptosis, exhaustion, differentiation, activation, inhibition or proliferation of the cell.

### BACKGROUND TO THE INVENTION

Adoptive immunotherapy of cancer involves the ex vivo generation of cancer-antigen specific cells and their administration. Two common methods for adoptive immunotherapy are using chimeric antigen receptors (CAR) or transgenic T-cell receptors (TCR). Different kinds of immune effector cells can also be used. For example, alpha/beta T-cells, NK cells, gamma delta T-cells or macrophages can be used.

Engineered cell therapy has been successful in treating a number of lymphoid malignancies, such as B-cell Acute Lymphoblastic Leukaemia (B-ALL), Diffuse Large B-cell Lymphoma (DLBCL) and Multiple Myeloma (MM), however there has been relatively little success in the treatment of solid cancers. There are many reasons why solid cancers have proven to be more difficult targets for engineered cell therapy than lymphoid cancers, including access to the tumour, persistence in the face of inhibitory signals and cells and heterogeneity of tumour antigen expression. Methods of increasing the potency of engineered cell therapies are needed for the successful treatment of cancers, such as solid cancers.

Accordingly, there remains a need for approaches to modulate the ability of engineered immune cells to function in a hostile microenvironment such as in a solid tumour. Such approaches may improve, for example, the ability of engineered immune cells to proliferate, survive and/or engraft in such a microenvironment.

### SUMMARY OF THE INVENTION

The present invention provides a cell which expresses an intracellular single domain protein binder which binds a target protein and modulates an intracellular signalling pathway.

The single domain protein binder may target a protein that functions in an intracellular signalling pathway which contributes to the activity and/or survival of the cell. For example, the single domain protein binder may target a protein which is involved in apoptosis, or a self-renewal, cell survival or cell activation pathway. Thus, the single domain protein binder may improve the ability of the cell to function in a hostile microenvironment by reducing its susceptibility to apoptosis, increasing the level of self-renewal and/or cell survival and/or increasing levels of cell activation (e.g. following antigen binding to a CAR or transgenic TCR).

Thus, in a first aspect the present invention provides a cell which expresses:

(i) an intracellular single domain protein binder which binds a target protein and modulates an intracellular signalling pathway; and
(ii) a chimeric antigen receptor (CAR) or a transgenic T cell receptor (TCR).

The single domain protein binder may be adomain antibody, for example, a single variable domain polypeptide (VHH) or a variable new antigen receptor (VNAR).

The single domain protein binder may be a non-antibody scaffold. It may have a molecular weight from about 6 kDa to about 20 kDa. For example, the non-antibody scaffold may be selected from an Affibody, Affilin, Anticalin, Atrimer, DARPin, FN3 scaffold, Fynomer, Pronectin and O-Body. In particular, the non-antibody scaffold may comprise a FN3 scaffold or a Fynomer.

The single domain protein binder may:

(i) reduce or prevent an interaction between the target protein and a second entity, such as a second polypeptide;
(ii) sequester and/or facilitate degradation of the target protein; or
(iii) activate the target protein.

The single domain protein binder may reduce or prevent an interaction between the target protein and a second entity by blocking or sterically hindering the binding site of the target protein for the second entity.

The single domain protein binder may comprise a degradation motif. It may target the target protein for proteasome-mediated degradation. The degradation motif may cause ubiquitation of the target protein. The degradation motif may, for example, comprises a sequence shown as one of SEQ ID NO: 21-23.

The intracellular signalling pathway may be involved in or cause apoptosis, exhaustion, differentiation, activation, inhibition or proliferation of the cell.

The intracellular signalling pathway may be SHP-2 mediated inhibition of cell activation. In this embodiment, the single domain protein binder may bind and/or block SHP-2. It may, for example, be or comprise an FN3 scaffold which binds SHP-2. Such as FN3 scaffold comprises a sequence shown as SEQ ID NO: 1 or 2.

The intracellular signalling pathway may be apoptosis. In this embodiment, the single domain protein binder may inhibit the activity of a proapoptotic signalling polypeptide such as FAS, FADD, P38, P53, CARD protein, BCL10, Capase 9, and Apaf1.

The single domain protein binder may be a FAS or FADD blocking polypeptide; or may target FAS or FADD for proteasome-mediated degradation.

The single domain protein binder may bind to the FAS cytoplasmic domain or the FADD Death domain.

The intracellular signalling pathway may be the TGFβ pathway. In this embodiment, the single domain protein binder may reduce or prevent a binding interaction between SMAD4 and the SMAD3/2 complex. The single domain protein binder be a SMAD4 blocking polypeptide. For example, it may bind and block the SMAD4 MH2 domain.

T cell may be a cytolytic immune cell such as a T-cell or natural killer (NK) cell.

In a second aspect, the present invention provides a nucleic acid construct which comprises: (i) a first nucleic acid sequence which encodes a single domain protein binder as defined in the first aspect of the invention; and (ii) a second nucleic acid sequence which encodes a chimeric antigen receptor (CAR) or a transgenic T cell receptor (TCR).

The nucleic acid sequences may be separated by a co-expression site.

In a third aspect, the present invention provides a kit of nucleic acid sequences comprising:

(i) a first nucleic acid sequence which encodes a single domain protein binder as defined in the first aspect of the invention; and
(ii) a second nucleic acid sequence which encodes a chimeric antigen receptor (CAR) or a transgenic T cell receptor (TCR).

In a fourth aspect, the present invention provides a vector which comprises a nucleic acid construct according to the second aspect of the invention.

In a fifth aspect, the present invention provides a kit of vectors which comprises:

(i) a first vector which comprises a nucleic acid sequence which encodes a single domain protein binder as defined in the first aspect of the invention; and
(ii) a second vector which comprises a nucleic acid sequence which encodes a chimeric antigen receptor (CAR) or a transgenic T cell receptor (TCR).

In a sixth aspect, the present invention provides a pharmaceutical composition which comprises a cell or plurality of cells according to the first aspect of the invention.

In a seventh aspect, the present invention provides a pharmaceutical composition according to the sixth aspect of the invention for use in treating and/or preventing a disease.

In an eighth aspect, the present invention provides a method for treating and/or preventing a disease, which comprises the step of administering a pharmaceutical composition according to the sixth aspect of the invention to a subject in need thereof.

The method according may comprise the following steps:

(i) isolation of a cell containing sample;
(ii) transduction or transfection of the cell with a nucleic acid construct according the second aspect of the invention; or a first nucleic acid sequence and a second nucleic acid sequence as defined in the third aspect of the invention, a vector according to the fourth aspect of the invention or a first and second vector as defined in the fifth aspect of the invention; and
(iii) administering the cells from (ii) to a subject.

In a ninth aspect, the present invention provides the use of a cell according to the first aspect of the invention in the manufacture of a medicament for the treatment and/or prevention of a disease.

The disease may be cancer.

In a tenth aspect, the present invention provides a method for making a cell according to the first aspect of the invention, which comprises the step of introducing into a cell with a nucleic acid construct according the second aspect of the invention; or a first nucleic acid sequence and a second nucleic acid sequence as defined in the third aspect of the invention, a vector according to the fourth aspect of the invention or a first and second vector as defined in the fifth aspect of the invention ex vivo.

The cell may be from a sample isolated from a subject.

### BRIEF DESCRIPTION OF THE FIGURES

FIG. 1—Diagram illustrating non-antibody scaffold proteins suitable for the present single domain protein binder

FIG. 2—Schematic diagram showing an illustrative approach for targeting TGFβ-mediated signalling via an single domain protein binder. In this illustrative embodiment an single domain protein binder (e.g. a single-domain antibody) binds to the SMAD4 MH2 domain and prevents SMAD4 complexing with SMAD2/3, thereby reducing SMAD2/3 mediated gene transcription.

FIG. 3—Schematic diagram showing an illustrative approach for targeting FAS/FADD (DISC) mediated pro-apoptotic signalling. In this illustrative embodiment an single domain protein binder binds to the FAS cytoplasmic domain or the FADD Death domain and prevents FAS complexing with FADD, thereby inhibiting caspase-mediated apoptosis.

FIG. 4—Schematic diagram showing an illustrative approach for targeting SHP-2 mediated inhibition of cell activation pathways. In this illustrative embodiment an single domain protein binder binds to the SH2 domain of SHP-2 and prevents SHP-2 dephosphorylating ITAM domains which mediate e.g. activation signalling from a CAR or transgenic TCR.

FIG. 5—Graph showing CD69 expression following co-culture of T cells expressing a GD2 CAR and optionally PD1 with a FN3 based SHP2 binder with target cells expressing GD2 and optionally PD-L1.

FIG. 6—Graph showing mean fluorescence of a BFP-expressing Raji cell, following transduction with construct expressing either a marker gene alone (RQR8) or coexpressing the marker gene with a BFP-targeting domain antibody fused to the U-box motif from e4B (VHH-U-box).

### DETAILED DESCRIPTION OF THE INVENTION

Single Domain Protein Binder

In a first aspect the present invention provides a cell which comprises an engineered intracellular binding polypeptide that is capable of modulating an intracellular signalling pathway. The engineered intracellular binding polypeptide is an intracellular single domain protein binder which binds a target protein and modulates an intracellular signalling pathway.

The single domain protein binder is encoded by a nucleic acid sequence which is not naturally encoded by the cell. Methods for engineering cells are known in the art and include but are not limited to genetic modification of cells e.g. by transduction such as retroviral or lentiviral transduction, transfection (such as transient transfection—DNA or RNA based) including lipofection, polyethylene glycol, calcium phosphate and electroporation. Any suitable method may be used to introduce a nucleic acid sequence into a cell.

The single domain protein binder should be capable of productive intracellular folding and specifically binding to a target protein in order to modulate an intracellular signalling pathway in the cell.

“Productive intracellular folding” is used herein to mean that the single domain protein binder is capable of folding into a conformation in which it is capable of specifically binding to its target protein within an intracellular environment (e.g. a reducing intracellular environment).

By way of example, conventional immunoglobulin molecules (e.g. entire IgGs) are incapable of correct folding in the reducing intracellular environment, in part because they require essential interchain disulfide bonds which do not form correctly in the intracellular environment and because the folding required for intracellular expression does not occur in the presence of appropriate chaperone proteins present in the endoplasmic reticulum.

The ability of a single domain protein binder to undergo productive intracellular folding may be determined using assays which are known in the art. Suitable assays include, but are not limited to co-immunoprecpitation, Forster Resonance Energy transfer (FRET) and Bioluminescence Resonance (BRET).

The ability of the single domain protein binder to specifically bind its target may be determined using methods which are known in the art. For example, determination of binding affinity (e.g. using a BIAcore instrument), western blot, flow cytometry, in situ hybridisation and/or microscopy.

Examples of polypeptides that may be used as single domain protein binder in the present invention include single-domain antibodies and a non-antibody scaffold polypeptides.

The single-domain protein binder consists essentially of a single protein domain. A domain is a distinct functional unit of a protein which comprises a compact three dimensional structure and can be independently stable and folded. For example, an IgG immunoglodulin is made up of two heavy chains and two light chains, each of which is composed of domains consisting of around 110 amino acid residues. The light chain has two domains (CL and VL), whereas the heavy chain has four (VH, CH1, CH2 and CH3). A single-chain variable fragment (scFv) has two domains (VH and VL) joined by a linker. A Fab fragment has four domains (VH, CH1, CL and VL).

The single domain protein binder may have a molecular weight of less than about 25 kDa or less than about 20 kDa. The single domain protein binder may have a molecular weight of about 5 kDa to about 25 kDa, about 5 kDa to about 20 kDa, about 10 kDa to about 25 kDa, about 10 kDa to about 20 kDa, or about 10 kDa to about 15 kDa.

A single-domain antibody (see below), also known as a nanobody, is an antibody fragment consisting of a single monomeric variable antibody domain. Domain antibodies or dAbs usually have a molecular weight of in the range 12-15 kDa.

Non-antibody scaffolds (see below) typically have a molecular weight between 5 and 25 kDa.

Suitably, the single domain protein binder is soluble in the intracellular environment.

In one aspect solubility of the single domain protein binder may be defined as the ability of the single domain protein binder to be purified, for example in phosphate buffered saline (PBS) (KCl 2.7 mM, KH₂PO₄ 1.5 mM, NaCl 137 mM and Na₂PO₄ 8 mM, pH 7.1-7.5. Life Technologies, Gibco BRL) at a concentration of 1 mg/ml and for 90% or more of said single domain protein binder to remain as a substantially monomeric after incubation at 25° C. for 1 week, 48 hours, 24 hours, 12 hours, or 1 hour.

The stability of the single domain protein binder in the intracellular environment may be determined using assays which are known in the art, for example differential scanning calorimetry and differential scanning fluorimetry.

In one aspect the single domain protein binder may have a melting temperature (Tm) which is greater than 50° C., greater than 55° C., greater than 60° C., greater than 65° C., greater than 70° C. or greater than 75° C.

Suitably, the single domain protein binder may not require the formation of a disulfide bond for functional folding. As used herein “functional folding” may refer to the folding of a single domain protein binder into a conformation in which it is capable of specifically binding to its target protein. “Functional folding” may refer to the folding of a single domain protein binder into a conformation in which it is soluble and/or stable in the intracellular environment. In other words, the single domain protein binder may comprise a disulfide bond, but the formation of this disulphide bond is not required for functional folding.

Suitably, the single domain protein binder may not comprise a disulfide bond.

Single-Domain Antibodies

The single domain protein binder may comprise a single-domain antibody (sdAb). sdAb are also referred to herein as “intrabodies”.

sdAbs are unique IgG molecules that are found naturally in e.g. camelids. Unlike conventional IgGs, sdAbs are devoid of the light chain and lack the first constant domain of the heavy chain. Consequently, the antigen-binding fragment of sdAbs is solely composed of a single variable domain, often referred to as a VHH. Cartilaginous fish also have heavy-chain antibodies (IgNAR, ‘immunoglobulin new antigen receptor’) from which single-domain antibodies called VNAR (variable new antigen receptor) fragments can be obtained.

sdAbs are endowed with favorable characteristics such as size, solubility and affinity. In addition, due to the lack of intra chain disulfide bonds, these antibody fragments have been shown to be capable of productive folding in the reducing intracellular environment. As described above, these properties make sdAbs suitable for targeting proteins within the cell.

sdAbs have a molecular weight of about 12 to about 15 kDa and are typically about 110 amino acids in length.

The single domain protein binder may comprise a VHH or VNAR.

The single domain protein binder may consist essentially of a VHH or a VNAR. In other words, the single domain protein binder may not comprise a hinge and/or constant region.

Methods for providing sdAbs against a specific target are known in the art (see Caussinus et al.; Nat Struct Mol Biol; 2011; 19(1); 117-121 & Fulcher et al.; Open Biol; 2016; 6(10); pii 160255—each of which is incorporated herein by reference). Further, methods to isolate antigen-specific VHHs from immune or semisynthetic libraries using phage, yeast, or ribosome display are established in the art (see Muyldermans J Biotechnol. 2001 June; 74(4):277-302. & Dufner et al. Trends Biotechnol. 2006 November; 24(11):523-9—each of which is incorporated herein by reference).

By way of example, a sdAb can be obtained by immunization of e.g. dromedaries, camels, llamas, alpacas or sharks with the desired antigen and subsequent isolation of the mRNA coding for heavy-chain antibodies. Reverse transcription and PCR can then be used to generate a library of sdAbs. Standard screening techniques such as phage display and ribosome display may be used to identify the suitable clones binding the antigen of interest.

Once the most potent clones have been identified, their DNA sequence may optimized, for example to improve their stability towards enzymes. Humanization may also be performed.

sdAbs may be expressed in a cell using conventional vectors, such as those described herein.

Non-Antibody Scaffolds

The single domain protein binder may be a non-antibody scaffold. As used herein, non-antibody scaffold refers to a binding polypeptide that does not bind to its target protein via complementary determining regions (CDRs).

The non-antibody scaffold may be a domain-sized scaffold. In particular, the non-antibody scaffold may be a domain-sized scaffold with a molecular weight from about 6 kDa to about 20 kDa.

Non-antibody scaffolds bind to a target protein via a range of different polypeptide conformational architectures which mediate protein-protein interactions.

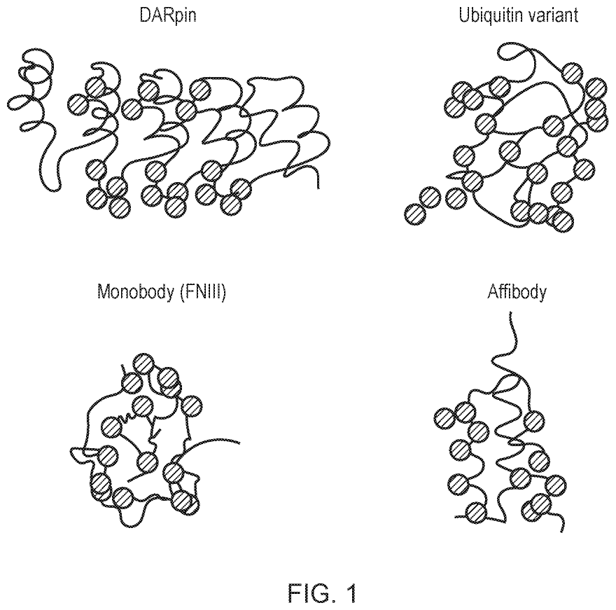

A summary of suitable, illustrative non-antibody scaffolds is shown in Table 2 and FIG. 1.

TABLE 2

| | | | | | Structural |
|---|---|---|---|---|---|
| | Parental | | Specificity | MW | disulfide |
| Scaffold | protein | Structure | Derivation | kPa | bond(s) |
| Affibodies | Z domain | α-helical | Helix | 6 | No |
| | | | randomization | | |
| Affilins | Ubiquitin | α/β | Beta strand | 10 | No |
| | | | randomization | | |
| Anticalins | Lipocalin | B Sheet α | Loop | 20 | No |
| | | helix | randomization | | |
| | | terminus | | | |
| Atrimers | C-type Lectin | α/β | Loop | 3 × 20 | No |
| | | | randomization | | |
| DARPins | Ankyrin | α helix + | Helix | 14-21 | No |
| | repeats | β turn | randomization | | |
| | | | β turn | | |
| | | | randomization | | |
| FN3 | Fibronectin | β Sheet | Loop | 10 | No |
| scaffolds | (type III) | | randomization | | |
| (Adnectins, | | | B strand | | |
| Centyrins, | | | randomization | | |
| Pronectins, | | | | | |
| Tn3) | | | | | |
| Fynomers | SH3 domain | β Sheet | Loop | 7 | No |
| | (fyn kinase) | | randomization | | |
| Kunitz | Serine | α/β | Loop | 7 | Yes |
| domains | protease | | randomization | | |
| | inhibitors | | | | |
| O-bodies | OB-fold | β Sheet | Loop | 12 | No |
| | | | randomization | | |
| | | | Beta sheet | | |
| | | | randomization | | |

The non-antibody scaffold domain may comprise an fibronectin type III (FN3) scaffold (e.g. Adnectins and Centyrins), Fynomer, Affibody, Affilin, Anticalin, Atrimer, DARPin, Pronectin or O-Body.

The non-antibody scaffold domain may comprise a FN3 scaffold.

FN3 scaffolds may be generated from combinatorial libraries in which portions of the FN3 scaffold are diversified using molecular display and directed evolution technologies such as phage display, mRNA display and yeast surface display. A large number of FN3 scaffolds that have high affinity and high specificity to their respective targets are known in the art. FN3 scaffolds have a structure similar to antibody variable domains, with seven beta sheets (referred to as A-G) forming a beta-sandwich and three exposed loops on each side corresponding to the three complementarity determining regions. By way of example, there are two distinct designs of FN3 libraries that have been successful. The first type modifies some or all of the loops BC (between the second and third beta sheets), DE (between the fourth and fifth beta sheets) and FG (between the sixth and seventh sheets). This design creates diversified positions on a convex surface that is suitable for targeting concave surfaces such as enzyme active sites. The second type modifies positions in some or all of the C, D, F and G (or the 3rd, 4th, 6th and 7th) strands in addition to the CD and FG loops. This design creates a flatter, slightly concave surface that is suitable for targeting surfaces typically involved in protein-protein interactions.

By way of example, the non-antibody scaffold may comprise a FN3 scaffold that is capable of specifically binding to the SH2 domain of Tyrosine-protein phosphatase non-receptor type 11 (PTPN11/SHP-2). Illustrative FN3 scaffolds that specifically bind to the SH2 domain of SHP-2 are described by Sha et al.; PNAS, 2013, vol. 110, no. 37—incorporated herein by reference. These FN3 scaffolds are capable of specifically binding to N terminal SH2 domain or C terminal SH2 domain of SHP-2, as summarised in Table 4. In Table 4, binders with a name starting “Nsa” bind the N-terminal SH2 domain, whereas binder with a name starting “Cs” bind the C-terminal SH2 domain.

TABLE 4
SHP2-binding FN3-based binding domains

| Name | Sequence |
|---|---|
| Nsa1 | SSVPTKLEVVAATPTSLLISWDAPAVTVDYYVITYGETGSGGYAWQEFEVPGSK |
| | STATISGLKPGVDYTITVYAGYYGYPTYYSSPISINYRT (SEQ ID NO: 1) |
| Nsa2 | SSVPTKLEVVAATPTSLLISWDAPAVTVDYYVITYGETGYYAYFQEFEVPGSKST |
| | ATISGLKPGVDYTITVYAGYYGYYGSPISINYRT (SEQ ID No: 26) |
| Nsa4 | SSVPTKLEVVAATPTSLLISWDAPAVTVDYYVITYGETGYYAYFQEFEVPGSKST |
| | ATISGLKPGVDYTITVYAGYYGYYGNPISINYRT (SEQ ID NO: 27) |
| Nsa5 | SSVPTKLEVVAATPTSLLISWDAPAVTVDFYVITYGETGYFSGFQEFEVPGSKST |
| | ATISGLKPGVDYTITVYAGYYGYYGSPISINYRT (SEQ ID NO: 2) |
| Cs1 | SSVPTKLEVVAATPTSLLISWDAPAVTVDYYVITYGETGYWPYYWQEFEVPGSK |
| | STATISGLKPGVDYTITVYAGSYDSYYYYGSPISINYRT (SEQ ID NO: 28) |
| Cs3 | SSVPTKLEVVAATPTSLLISWDAPAVTVDYYVITYGETGHWPWVWQEFEVPGSK |
| | STATISGLKPGVDYTITVYAGSYSSYYYYGSPISINYRT (SEQ ID NO: 29) |
| Cs4 | SSVPTKLEVVAATPTSLLISWDAPAVTVDYYVITYGETGYWPYYWQEFEVPGSK |
| | STATISGLKPGVDYTITVYAGHYNSYYYSYSPISINYRT (SEQ ID NO: 33) |

The FN3 scaffold may comprise one of the amino acid sequence shown in Table 4 or a variant thereof. Variant sequences may have at least 80%, 85%, 90%, 95%, 98% or 99% sequence identity to a sequence shown in Table 4, provided that the FN3 is capable of specifically binding an SH2 domain of SHP-2.

The FN3 scaffold may comprise two or more sequences shown in table 4, joined by a linker. It may comprise a binder which binds the N-terminal SH2 domain of SHP2 linked to a binder which binds the C-terminal SH2 domain of SHP2. For example, it may comprise Nsa5 linked to Cs3 in either orientation.

Each sequence comprises a CD loop and FG loop, as shown below for Nsa1 and Nsa5.

(Nsa1)
SEQ ID NO: 1
SSVPTKLEVVAATPTSLLISWDAPAVTVDYYVITYGETGSGGYAWQEFE
VPGSKSTATISGLKPGVDYTITVYAGYYGYPTYYSSPISINYRT
CD Loop:
(SEQ ID NO: 3)
SGGYAW
FG Loop:
(SEQ ID NO: 4)
AGYYGYPTYYSS
(Nsa5)
SEQ ID NO: 2
SSVPTKLEVVAATPTSLLISWDAPAVTVDFYVITYGETGYFSGFQEFE
VPGSKSTATISGLKPGVDYTITVYAGYYGYYGSPISINYRT
CD Loop:
(SEQ ID NO: 5)
YFSGF
FG Loop:
(SEQ ID NO: 6)
AGYYGYYGS

The non-antibody scaffold domain may comprise a Fynomer scaffold.

Intracellular Signalling Pathway

The single domain protein binder may be capable of specifically binding and modulating any protein which is part of an intracellular signalling pathway that contributes to the functioning of the cell of the present invention.

By “modulating a protein” it is meant that the single domain protein binder increases or inhibits/reduces the activity of the target protein. As will be apparent, the increase or inhibition/reduction in activity refers to a comparison to the activity in a corresponding, control cell which is treated in identical conditions to the cell of invention, except that it does not comprise an single domain protein binder as described herein.

For example, the single domain protein binder may specifically bind and modulate the activity of a protein which is part of an apoptosis, self-renewal, cell survival or cell activation intracellular pathway.

Thus, binding of the single domain protein binder to its target protein may result in an improvement in the ability of the present engineered immune cell to function in a hostile microenvironment by e.g. reducing its susceptibility to apoptosis, increasing its level of self-renewal and/or cell survival and/or increasing the level of cell activation (e.g. following antigen binding to a CAR or transgenic TCR).

Apoptotic Pathways

Apoptotic pathways are well-known in the art and are typically categorised as the intrinsic pathway or the extrinsic pathway. The intrinsic pathway is activated by intracellular signals generated when cells are stressed and depends on the release of proteins from the intermembrane space of mitochondria. The extrinsic pathway is activated by extracellular ligands binding to cell-surface death receptors, which leads to the formation of the death-inducing signalling complex (DISC).

The single domain protein binder may inhibit/reduce the activity of a pro-apoptotic protein or increase the activity of an anti-apoptotic protein. Suitably, the single domain protein binder inhibits/reduces the activity of a pro-apoptotic protein.

By way of example, the pro-apoptotic polypeptide may be selected from Fas, FASS, P38, P53, CARD protein, BCL10, Capase 9, and Apaf1.

As used herein, inhibiting the activity of a pro-apoptotic polypeptide may refer to a reduction in the activity of the pro-apoptotic protein by at least 10%, at least 20%, at least 30%, at least 40%, at least 50%, at least 60%, at least 70%, at least 80%, at least 90%, at least 95% or at least 99%.

As will be apparent, the reduction in activity refers to a comparison to the activity in a corresponding, control cell which is treated in identical conditions to the cell of invention, except that it does not comprise an engineered single domain protein binder as described herein.

The extrinsic apoptosis pathway in mammals may be stimulated by the TNF-induced (tumor necrosis factor) pathway or the Fas-Fas ligand-mediated pathway, both of which involve receptors of the TNF receptor (TNFR) family coupled to extrinsic signals.

The single domain protein binder may bind to a target protein which is part of the Fas, TNFα and/or TGF6 induced apoptotic signalling pathway.

Fas Pathway

The Fas receptor (also known as Apo-1 or CD95) is a transmembrane protein of the TNF family which binds the Fas ligand (FasL). The interaction between Fas and FasL results in the formation of the death-inducing signaling complex (DISC), which contains FADD, caspase-8 and caspase-10. Multimerization of intracellular FADD domains is required for proper DISC formation. The multimerization of intracellular FADD polypeptides is mediated by interactions between Death Domains and Death Effector Domains of FADD. In some types of cells (type I), processed caspase-8 directly activates other members of the caspase family, and triggers the execution of apoptosis of the cell. In other types of cells (type II), the Fas-DISC starts a feedback loop that spirals into increasing release of proapoptotic factors from mitochondria and the amplified activation of caspase-8.

The single domain protein binder may specifically bind to a pro-apoptotic protein in the Fas pathway and inhibit activity of the pro-apoptotic polypeptide.

For example, the single domain protein binder may specifically bind to Fas, FADD, caspase-8 or caspase-10.

The formation of the DISC is mediated by an interaction between the cytoplasmic domain of Fas and the Death-domain of FADD.

The single domain protein binder may specifically bind to the intracellular domain of Fas or the Death-domain of FADD and inhibit the interaction between Fas and FADD.

The single domain protein binder may specifically bind to FADD to reduce or prevent multimerization of FADD polypeptides (i.e. to inhibit the interaction between two or more FADD polypeptides). The single domain protein binder may specifically bind to the Death Domain and/or Death effector domain of FADD.

An illustrative, human Fas amino acid sequence is provided by Uniprot Accession Number: P25445 and shown as SEQ ID NO: 7.

SEQ ID NO: 7
MLGIWTLLPLVLTSVARLSSKSVNAQVTDINSKGLELRKTVTT
VETQNLEGLHHDGQFCHKPCPPGERKARDCTVNGDEPDCVPCQ
EGKEYTDKAHFSSKCRRCRLCDEGHGLEVEINCTRTQNTKCRC
KPNFFCNSTVCEHCDPCTKCEHGIIKECTLTSNTKCKEEGSRS
NLGWLCLLLLPIPLIVWVKRKEVQKTCRKHRKENQGSHESPTL
NPETVAINLSDVDLSKYITTIAGVMTLSQVKGFVRKNGVNEAK
IDEIKNDNVQDTAEQKVQLLRNWHQLHGKKEAYDTLIKDLKKA
NLCTLAEKIQTIILKDITSDSENSNFRNEIQSLV

An illustrative sequence for the cytoplasmic domain of Fas is shown as SEQ ID NO: 8.

SEQ ID NO: 8
KRKEVQKTCRKHRKENQGSHESPTLNPETVAINLSDVDLSKYI
TTIAGVMTLSQVKGFVRKNGVNEAKIDEIKNDNVQDTAEQKVQ
LLRNWHQLHGKKEAYDTLIKDLKKANLCTLAEKIQTIILKDIT
SDSENSNFRNEIQSLV

An illustrative, human FADD amino acid sequence is provided by Uniprot Accession Number: Q13158 and shown as SEQ ID NO: 9.

SEQ ID NO: 9
MDPFLVLLHSVSSSLSSSELTELKFLCLGRVGKRKLERVQSGL
DLFSMLLEQNDLEPGHTELLRELLASLRRHDLLRRVDDFEAGA
AAGAAPGEEDLCAAFNVICDNVGKDWRRLARQLKVSDTKIDSI
EDRYPRNLTERVRESLRIWKNTEKENATVAHLVGALRSCQMNL
VADLVQEVQQARDLQNRSGAMSPMSWNSDASTSEAS

The Death effector domain of FADD corresponding to amino acid positions 3-81 of the sequence shown as SEQ ID NO: 9. The Death domain of FADD corresponds to amino acid positions 97-181 of the sequence shown as SEQ ID NO: 9.

Caspase binding to FADD occurs through a binding region in the Death Effector Domain of FADD. The single domain protein binder may specifically bind to the Death Effector Domain of FADD to reduce or prevent interaction between FADD and caspases.

Suitable assays for determining whether the interaction between Fas and FADD, FADD multimerization and/or FADD and caspase is inhibited are known in the art and include, for example inhibition of the molecular interactions through phospho-serine western blotting experiments on cell lysate directed to S194 on FADD and the presence of cleaved Caspases. Inhibition of apoptosis after activation of the FAS signalling pathway can be measured by 7AAD and Annexin V staining using flow cytometry.

TNF Pathway

TNFα is a cytokine produced mainly by activated macrophages. Most cells in the human body have two receptors for TNF-alpha: TNFR1 and TNFR2. The binding of TNF-alpha to TNFR1 has been shown to initiate the pathway that leads to caspase activation via the intermediate membrane proteins TNF receptor-associated death domain (TRADD) and Fas-associated death domain protein (FADD).

An illustrative, human TNFR1 amino acid sequence is provided by Uniprot Accession Number: P19438 and shown as SEQ ID NO: 10.

SEQ ID NO: 10
MGLSTVPDLLLPLVLLELLVGIYPSGVIGLVPHLGDREKRDSV
CPQGKYIHPQNNSICCTKCHKGTYLYNDCPGPGQDTDCRECES
GSFTASENHLRHCLSCSKCRKEMGQVEISSCTVDRDTVCGCRK
NQYRHYWSENLFQCFNCSLCLNGTVHLSCQEKQNTVCTCHAGF
FLRENECVSCSNCKKSLECTKLCLPQIENVKGTEDSGTTVLLP
LVIFFGLCLLSLLFIGLMYRYQRWKSKLYSIVCGKSTPEKEGE
LEGTTTKPLAPNPSFSPTPGFTPTLGFSPVPSSTFTSSSTYTP
GDCPNFAAPRREVAPPYQGADPILATALASDPIPNPLQKWEDS
AHKPQSLDTDDPATLYAVVENVPPLRWKEFVRRLGLSDHEIDR
LELQNGRCLREAQYSMLATWRRRTPRREATLELLGRVLRDMDL
LGCLEDIEEALCGPAALPPAPSLLR

The cytoplasmic domain of TNFR1 corresponds to amino acid positions 233-455 of the sequence shown as SEQ ID NO: 10.

The single domain protein binder may specifically bind to the cytoplasmic domain of TNFR1 and inhibit the interaction between TNFR1, TRADD and/or FADD.

An illustrative, human TRADD amino acid sequence is provided by Uniprot Accession Number: Q15628 and shown as SEQ ID NO: 11.

SEQ ID NO: 11
MAAGQNGHEEWVGSAYLFVESSLDKVVLSDAYAHPQQKVAVYR
ALQAALAESGGSPDVLQMLKIHRSDPQLIVQLRFCGRQPCGRF
LRAYREGALRAALQRSLAAALAQHSVPLQLELRAGAERLDALL
ADEERCLSCILAQQPDRLRDEELAELEDALRNLKCGSGARGGD
GEVASAPLQPPVPSLSEVKPPPPPPPAQTFLFQGQPVVNRPLS
LKDQQTFARSVGLKWRKVGRSLQRGCRALRDPALDSLAYEYER
EGLYEQAFQLLRRFVQAEGRRATLQRLVEALENELTSLAEDLL
GLTDPNGGLA

The Death domain of TRADD corresponds to amino acid positions 179-289 of the sequence shown as SEQ ID NO: 11.

The single domain protein binder may specifically bind to the Death domain of TRADD and inhibit the interaction between TNFR1 and FADD.

Common Components

Caspases play the central role in the transduction of ER apoptotic signals. Caspases are proteins that are highly conserved, cysteine-dependent aspartate-specific proteases. There are two types of caspases: initiator caspases, caspase 2,8,9,10,11,12, and effector caspases, caspase 3,6,7. The activation of initiator caspases requires binding to specific oligomeric activator protein. Effector caspases are then activated by these active initiator caspases through proteolytic cleavage. The active effector caspases then proteolytically degrade a host of intracellular proteins to carry out the cell death program.

The single domain protein binder may specifically bind to and inhibit a caspase polypeptide.

Fas-associated death domain-like interleukin-1-β-converting enzyme-inhibitory protein long form (FLIP_{L}), is a potent inhibitor of death receptor-induced apoptosis. In particular, FLIP_{L} directly competes with procaspases (e.g. caspase-8) to inhibit formation of active homodimers.

An illustrative, human FLIP_{L} amino acid sequence is provided by Uniprot Accession Number: 015519 and shown as SEQ ID NO: 25.

SEQ ID NO: 25
MSAEVIHQVEEALDTDEKEMLLFLCRDVAIDVVPPNVRDLLDI
LRERGKLSVGDLAELLYRVRRFDLLKRILKMDRKAVETHLLRN
PHLVSDYRVLMAEIGEDLDKSDVSSLIFLMKDYMGRGKISKEK
SFLDLVVELEKLNLVAPDQLDLLEKCLKNIHRIDLKTKIQKYK
QSVQGAGTSYRNVLQAAIQKSLKDPSNNFRLHNGRSKEQRLKE
QLGAQQEPVKKSIQESEAFLPQSIPEERYKMKSKPLGICLIID
CIGNETELLRDTFTSLGYEVQKFLHLSMHGISQILGQFACMPE
HRDYDSFVCVLVSRGGSQSVYGVDQTHSGLPLHHIRRMFMGDS
CPYLAGKPKMFFIQNYVVSEGQLEDSSLLEVDGPAMKNVEFKA
QKRGLCTVHREADFFWSLCTADMSLLEQSHSSPSLYLQCLSQK
LRQERKRPLLDLHIELNGYMYDWNSRVSAKEKYYVWLQHTLRK
KLILSYT

The present invention also provides a cell which expresses a chimeric antigen receptor (CAR) or a transgenic T cell receptor (TCR) and over-expresses FLIP_{L}.

The cell may comprise an engineered polynucleotide encoding a FLIP_{L} polypeptide. An engineered polynucleotide refers to a polynucleotide which is not naturally present in the endogenous, genomic DNA of the cell. For example, the engineered polynucleotide may be a vector.

The FLIP_{L} polypeptide may comprise an amino acid sequence of SEQ ID NO: 25, or a variant with at least 80, 85, 90, 95 or 99% sequence identity to SEQ ID NO: 25. The variant of SEQ ID NO: 25 should maintain the ability to inhibit death receptor-induced apoptosis.

Suitably, the FLIP_{L} polypeptide may further comprise a degradation motif as described herein, such that it is capable of targeting FADD and/or a caspase (e.g. caspase-8) for ubiquitin-mediated degradation.

Aspect of the invention relating to nucleic acid constructs, vectors, kits of nucleic acid sequences, kits of vectors, pharmaceutical compositions and uses thereof, methods of treatment and method of making the cells of the invention apply equally to cells which co-express a CAR or engineered TCR and overexpress FLIP_{L}.

TGFβ Signalling Pathway

The single domain protein binder may specifically bind and inhibit the activity of a proapoptotic protein in the Transforming growth factor beta (TGFβ) signalling pathway.

TGFβ is a cytokine belonging to the transforming growth factor superfamily. TGFβ receptors are a superfamily of serine/threonine kinase receptors. These receptors bind members of the TGFβ superfamily of growth factor and cytokine signalling proteins. There are five type II receptors (which are activatory receptors) and seven type I receptors (which are signalling propagating receptors). Type I receptors are also known as activin receptor-like kinases (ALKS). TGFβ1, 2 and 3 signal via binding to receptors TβR11 and then association to TβRI and in the case of TGFβ2 also to TβRIII. This leads to subsequent signalling through SMADs via WI. Binding of TGFβ to WI and TβR11 results in phosphorylation of WI and recruitment of the SMAD2/3 complex, which subsequently interacts with SMAD4 before translocation to the nucleus and induction of TGFβ-responsive gene activation. This gene activation includes expression of e.g. death-associated protein kinase (DAP-kinase) as an immediate early response in cells that undergo apoptosis in response to TGF-β.

The single domain protein binder may specifically bind and inhibit the activity of TβRI, TβRII, SMAD2/3 or SMAD4.

For example, the single domain protein binder may specifically bind SMAD2/3 or SMAD4 and reduce or prevent a binding interaction between SMAD4 and the SMAD3/2 complex.

The single domain protein binder may specifically bind the SMAD4 MH2 domain and reduce of prevent the binding interaction between SMAD4 and the SMAD3/2 complex.

An illustrative, human SMAD4 amino acid sequence is provided by Uniprot Accession Number: Q13485 and shown as SEQ ID NO: 12.

SEQ ID NO: 12
MDNMSITNTPTSNDACLSIVHSLMCHRQGGESETFAKRAIESL
VKKLKEKKDELDSLITAITTNGAHPSKCVTIQRTLDGRLQVAG
RKGFPHVIYARLWRWPDLHKNELKHVKYCQYAFDLKCDSVCVN
PYHYERVVSPGIDLSGLTLQSNAPSSMMVKDEYVHDFEGQPSL
STEGHSIQTIQHPPSNRASTETYSTPALLAPSESNATSTANFP
NIPVASTSQPASILGGSHSEGLLQIASGPQPGQQQNGFTGQPA
TYHHNSTTTWTGSRTAPYTPNLPHHQNGHLQHHPPMPPHPGHY
WPVHNELAFQPPISNHPAPEYWCSIAYFEMDVQVGETFKVPSS
CPIVTVDGYVDPSGGDRFCLGQLSNVHRTEAIERARLHIGKGV
QLECKGEGDVWVRCLSDHAVFVQSYYLDREAGRAPGDAVHKIY
PSAYIKVFDLRQCHRQMQQQAATAQAAAAAQAAAVAGNIPGPG
SVGGIAPAISLSAAAGIGVDDLRRLCILRMSFVKGWGPDYPRQ
SIKETPCWIEIHLHRALQLLDEVLHTMPIADPQPLD

The MH2 domain of SMAD4 is shown as SEQ ID NO: 13.

SEQ ID NO: 13
WCSIAYFEMDVQVGETFKVPSSCPIVTVDGYVDPSGGDRFCLG
QLSNVHRTEAIERARLHIGKGVQLECKGEGDVWVRCLSDHAVF
VQSYYLDREAGRAPGDAVHKIYPSAYIKVFDLRQCHRQMQQQA
ATAQAAAAAQAAAVAGNIPGPGSVGGIAPAISLSAAAGIGVDD
LRRLCILRMSFVKGWGPDYPRQSIKETPCWIEIHLHRALQLLD
EVLHTMPIADPQPLD

Suitable assays for determining whether the interaction between SMAD4 and SMAD2/3 is inhibited are known in the art and include, for example immunoprecipitation of cell lysate for SMAD4 followed by a western blot of SMAD2/3. Functional inhibition of the signalling pathway after ligand engagement can be measured by flow cytometry on e.g. T-cells after co-culture on targets that express the cognate ligand and soluble TGFβ1.

Self-Renewal Signalling Pathways

Self-renewal is used herein to refer to the ability of a cell according to the present invention to proliferate and expand. In particular, self-renewal is used herein to refer to the ability of a cell according to the present invention to proliferate and expand within the tumour microenvironment.

Intracellular signalling pathways that stimulate a cell to undergo cell division and proliferation are well-known in the art and include, for example, those shown in Table 3.

TABLE 3

| | Intracellular | Pathway Inhibitors |
|---|---|---|
| | Signalling Molecule/ | (anti-proliferative |
| Pathway | Transcription Factor | molecules) |
| Mitogens | MAPK/Jun/Fos | |
| Cytokines | STAT3 | |
| Extracellular Matrix | KLF | Jumonji |
| Notch | CSL | |
| Wnt | B-catenin/TCF | β-TRCP |
| Immune Signals | NF-κB | Iκβ/SIP |

The single domain protein binder may specifically bind to and inhibit an anti-proliferative molecule that inhibits a proliferation pathway (e.g. a pathway shown in Table 3).

As used herein, inhibiting the activity may refer to a reduction in the activity of the anti-proliferative protein by at least 10%, at least 20%, at least 30%, at least 40%, at least 50%, at least 60%, at least 70%, at least 80%, at least 90%, at least 95% or at least 99% compared to the activity in the absence of the single domain protein binder.

The single domain protein binder may specifically bind to and inhibit IκB (inhibitor for the transcription factor NFκB). The single domain protein binder may bind IκB at the same binding interface as NFκB and release NFκB and allow it to be active.

An illustrative, human IκB amino acid sequence is provided by Uniprot Accession Number: P25963 and shown as SEQ ID NO: 14.

SEQ ID NO: 14
MFQAAERPQEWAMEGPRDGLKKERLLDDRHDSGLDSMKDEEYE
QMVKELQEIRLEPQEVPRGSEPWKQQLTEDGDSFLHLAIIHEE
KALTMEVIRQVKGDLAFLNFQNNLQQTPLHLAVITNQPEIAEA
LGAGCDPELRDFRGNTPLHLACEQGCLASVGVLTQSCTTPHLH
SILKATNYNGHTCLHLASIHGYLGIVELLVSLGADVNAQEPCN
GRTALHLAVDLQNPDLVSLLLKCGADVNRVTYQGYSPYQLTWG
RPSTRIQQQLGQLTLENLQMLPESEDEESYDTESEFTEFTEDE
LPYDDCVFGGQRLTL

IκB binds to NFκB via a six-ankyrin repeat domain (ARD) (shown as amino acid positions 67-287 of SEQ ID NO: 14). The single domain protein binder may specifically bind to IκB via an epitope within the amino acid sequence which corresponds to amino acid positions 67-287 of SEQ ID NO: 14 and thereby inhibit IκB binding to NFκB.

The single domain protein binder may specifically bind to and inhibit a negative regulator which targets an intracellular signalling molecule or transcription factor for degradation, for example ubiquitin/proteasome-mediated degradation.

For example, β-catenin is a downstream signalling protein in the Wnt pathway that is involved in the transcription factor process. β-catenin is regulated through degradation which is mediated by β-TRCP. The single domain protein binder may specifically bind to and inhibit β-TRCP such that β-catenin is released and can be active.

The capacity of a cell to undergo proliferation is typically in opposition to its ability to differentiate. In other words, a cell will typically undergo proliferation and divisional or undergo differentiation; but not both simultaneously.

Accordingly, the single domain protein binder may specifically bind to and inhibit a transcription factor that simulates cell differentiation (e.g. T cell differentiation).

Transcription factors that are important for T-cell differentiation include, but are not limited to, Runx, T-bet, GATS, Foxp3, RORγt and EOMES. The single domain protein binder may bind to the DNA binding interface of the transcription factor and inhibit the ability of the transcription factor to induce cell differentiation.

Methods for determining inhibition of cell differentiation (e.g. T cell differentiation) are known in the art and include, for example, flow cytometric analysis of cell surface markers can be used to monitor T cell differentiation. For example, analysis of cell surface markers CCR7 and CD45RA may be monitored. The following expression patterns may be used to evaluate T cell differentiation Naive: CCR7+/CD45RA+, Central memory: CCR7-CD45RA−, Effector memory: CCR7-/CD45Ra−, Effector: CCR7-/CD45RA+.

The single domain protein binder may specifically bind to a transmembrane receptor, intracellular signalling molecule or transcription factor (e.g. as listed in Table 3) and induce and/or increase the activity of the transmembrane receptor, intracellular signalling molecule or transcription factor in order to increase proliferation/self-renewal of a cell according to the present invention.

Suitably, the single domain protein binder may cause activation of its target protein through oligomerisation.

For example, a number of transmembrane receptors require homodimerization for activation (e.g. receptor tyrosine kinases such as C-MYC, FLT-3). The single domain protein binder may bind to at least two such target proteins in order to induce homodimerzation/oligomerization and subsequent activation of the protein.

The single domain protein binder may be capable of binding to two, three, four, five or more target proteins.

The single domain protein binder may be capable of binding to two, three, or four target proteins.

The single domain protein binder may bind to a plurality of the same target protein. In other words, the single domain protein binder may be capable of inducing homodimerization or homomultimerization of its target protein.

The single domain protein binder may bind to one or more different target proteins. In other words, the single domain protein binder may be capable of inducing heterodimerization or heteromultimerization of its target proteins

Binding of multivalent single domain protein binders to a plurality of target proteins (for example, comprising coiled coil structures) may result in the generation of higher order aggregates of these target proteins (e.g. receptors on the cell membrane) resulting in super activation of the target protein.

The Wnt1 signalling pathway is activated through the bridging of the two transmembrane proteins Frizzed and LRP. The single domain protein binder may bind to Frizzed and LRP by a bivalent interaction and activate Wnt1 signalling in the cell.

A number of transcription factors require dimerization of separate subunits in order to activate gene expression (e.g. AP1 and STATs).

The single domain protein binder may bind to the subunits of a dimeric transcription factor in order to induce dimerization and activation of the transcription factor. By way of example, the single domain protein binder may be a bivalent molecule that binds to and induces dimerization of FOS and JUN (for AP1) or STAT pairs through bivalent interactions.

The single domain protein binder may bind to a target protein and cause it to be activated by localising it to an appropriate subcellular location.

For example, the single domain protein binder may comprise a membrane tethering component and a binding domain which binds to a target protein that is activated following localisation to the cell membrane.

By way of example, AKT is an intracellular kinase that requires localisation to the membrane for activation. The single domain protein binder may comprise a membrane tethering component and a binding domain which is capable of specifically binding AKT.

Membrane Tethering Component

Suitable membrane tethering components are known in the art and act as an anchor, tethering the protein to the cell membrane.

The membrane tethering component may comprise a membrane localisation domain. This may be any sequence which causes the protein to be attached to or held in a position proximal to the plasma membrane. The membrane localisation domain may be or comprise a sequence which causes the nascent polypeptide to be attached initially to the ER membrane. As membrane material “flows” from the ER to the Golgi and finally to the plasma membrane, the protein remains associated with the membrane at the end of the synthesis/translocation process.

The membrane localisation domain may, for example, comprise a transmembrane domain or transmembrane sequence, a stop transfer sequence, a GPI anchor or a myristoylation/prenylation/palmitoylation site.

Alternatively the membrane localisation domain may direct the membrane-tethering component to a protein or other entity which is located at the cell membrane, for example by binding the membrane-proximal entity. The membrane tethering component may, for example, comprise a domain which binds a molecule which is involved in the immune synapse, such as TCR/CD3, CD4 or CD8.

In embodiment where the single domain protein binder its targeted to the membrane by a post-translation modification (e.g. myristoylation/prenylation/palmitoylation), the modification motif may be located at the N-terminus of the single domain protein binder in order to direct correct orientation of the binding polypeptide at the cell membrane (e.g. such that the binding domain is located on the intracellular side of the cell membrane).

Survival Signalling Pathways

As used herein, survival signalling pathways is used to refer to co-stimulatory signals which transmit a survival signal which reduces the propensity of the immune cell to be exhausted following antigen-binding and transmittal of Signal 1 (activating signal) alone.

Survival signalling pathways in immune cells are well-known in the art. For example, co-receptors such as 4-1BB (also known as CD137), CD28 and OX40 to transmit a survival signal via their respective immunoreceptor tyrosine-based activation motif (ITAM).

Co-receptors typically require dimerization or trimerization for activation. Accordingly, the single domain protein binder may bind to at least two target co-receptors polypeptides in order to induce oligomerization and subsequent activation of the co-receptors protein.

The single domain protein binder may be capable of binding to two, three, four, five or more target proteins.

Cell Activation Signalling

Activation of immune cells (e.g. T cells) depends on a balance between activating and inhibitory signalling.

After antigen recognition, receptors (e.g. CAR or TCRs) cluster, native CD45 and CD148 are excluded from the synapse and an activating signal is transmitted to the cell. Activating receptors (e.g. CD3-zeta, CD3-gamma, CD3-delta, 41-BB, CD28 and OX40) comprise endodomains which comprise one or more ITAM motifs.

Inhibitory signalling molecules (e.g. CD45, CD148, protein-tyrosine phosphatase such as PTPN6 (SHP-1) and PTPN11 (SHP-2)) typically comprise a domain with an Immunoreceptor Tyrosine-based Inhibition motif (ITIM) which provides a tyrosine phosphatase activity that is able to dephosphorylate an ITAM. An inhibitory endodomain may be or comprise any tyrosine phosphatase with a sufficiently fast catalytic rate for phosphorylated ITAMs that is capable of inhibiting CAR/TCR signalling.

CD148 is a receptor-like protein tyrosine phosphatase which negatively regulates TCR signaling by interfering with the phosphorylation and function of PLCy1 and LAT.

CD45 present on all hematopoetic cells, is a protein tyrosine phosphatase which is capable of regulating signal transduction and functional responses, again by phosphorylating PLC yl.

The transmembrane and endodomains of CD45 and CD148 are shown as SEQ ID NO: 15 and NO: 16 respectively.

CD45 trans-membrane and endodomain sequence
SEQ ID NO: 15
ALIAFLAFLIIVTSIALLVVLYKIYDLHKKRSCNLDEQQELVE
RDDEKQLMNVEPIHADILLETYKRKIADEGRLFLAEFQSIPRV
FSKFPIKEARKPFNQNKNRYVDILPYDYNRVELSEINGDAGSN
YINASYIDGFKEPRKYIAAQGPRDETVDDFWRMIWEQKATVIV
MVTRCEEGNRNKCAEYWPSMEEGTRAFGDVVVKINQHKRCPDY
IIQKLNIVNKKEKATGREVTHIQFTSWPDHGVPEDPHLLLKLR
RRVNAFSNFFSGPIVVHCSAGVGRTGTYIGIDAMLEGLEAENK
VDVYGYVVKLRRQRCLMVQVEAQYILIHQALVEYNQFGETEVN
LSELHPYLHNMKKRDPPSEPSPLEAEFQRLPSYRSWRTQHIGN
QEENKSKNRNSNVIPYDYNRVPLKHELEMSKESEHDSDESSDD
DSDSEEPSKYINASFIMSYWKPEVMIAAQGPLKETIGDFWQMI
FQRKVKVIVMLTELKHGDQEICAQYWGEGKQTYGDIEVDLKDT
DKSSTYTLRVFELRHSKRKDSRTVYQYQYTNWSVEQLPAEPKE
LISMIQVVKQKLPQKNSSEGNKHHKSTPLLIHCRDGSQQTGIF
CALLNLLESAETEEVVDIFQVVKALRKARPGMVSTFEQYQFLY
DVIASTYPAQNGQVKKNNHQEDKIEFDNEVDKVKQDANCVNPL
GAPEKLPEAKEQAEGSEPTSGTEGPEHSVNGPASPALNQGS
CD148 trans-membrane and endodomain sequence
SEQ ID NO: 16
AVFGCIFGALVIVTVGGFIFWRKKRKDAKNNEVSFSQIKPKKS
KLIRVENFEAYFKKQQADSNCGFAEEYEDLKLVGISQPKYAAE
LAENRGKNRYNNVLPYDISRVKLSVQTHSTDDYINANYMPGYH
SKKDFIATQGPLPNTLKDFWRMVWEKNVYAIIMLTKCVEQGRT
KCEEYWPSKQAQDYGDITVAMTSEIVLPEWTIRDFTVKNIQTS
ESHPLRQFHFTSWPDHGVPDTTDLLINFRYLVRDYMKQSPPES
PILVHCSAGVGRTGTFIAIDRLIYQIENENTVDVYGIVYDLRM
HRPLMVQTEDQYVFLNQCVLDIVRSQKDSKVDLIYQNTTAMTI
YENLAPVTTFGKTNGYIA

Protein tyrosine phosphatases (PTPs) are signaling molecules that regulate a variety of cellular processes including cell growth, differentiation, mitotic cycle, and oncogenic transformation. The N-terminal part of this PTP contains two tandem Src homolog (SH2) domains, which act as protein phospho-tyrosine binding domains, and mediate the interaction of this PTP with its substrates. This PTP is expressed primarily in hematopoietic cells, and functions as an important regulator of multiple signaling pathways in hematopoietic cells.

Illustrative amino acid sequences for PTPN6 (SEQ ID NO: 17) and the PTPN6 phosphatase domain (SEQ ID NO: 18) are shown below.

sequence of PTPN6
SEQ ID NO: 17
MVRWFHRDLSGLDAETLLKGRGVHGSFLARPSRKNQGDFSLS
VRVGDQVTHIRIQNSGDFYDLYGGEKFATLTELVEYYTQQQG
VLQDRDGTIIHLKYPLNCSDPTSERWYHGHMSGGQAETLLQA
KGEPWTFLVRESLSQPGDFVLSVLSDQPKAGPGSPLRVTHIK
VMCEGGRYTVGGLETFDSLTDLVEHFKKTGIEEASGAFVYLR
QPYYATRVNAADIENRVLELNKKQESEDTAKAGFWEEFESLQ
KQEVKNLHQRLEGQRPENKGKNRYKNILPFDHSRVILQGRDS
NIPGSDYINANYIKNQLLGPDENAKTYIASQGCLEATVNDFW
QMAWQENSRVIVMTTREVEKGRNKCVPYWPEVGMQRAYGPYS
VTNCGEHDTTEYKLRTLQVSPLDNGDLIREIWHYQYLSWPDH
GVPSEPGGVLSFLDQINQRQESLPHAGPIIVHCSAGIGRTGT
IIVIDMLMENISTKGLDCDIDIQKTIQMVRAQRSGMVQTEAQ
YKFIYVAIAQFIETTKKKLEVLQSQKGQESEYGNITYPPAMK
NAHAKASRTSSKHKEDVYENLHTKNKREEKVKKQRSADKEKS
KGSLKRK
sequence of phosphatase domain of PTPN6
SEQ ID NO: 18
FWEEFESLQKQEVKNLHQRLEGQRPENKGKNRYKNILPFDHS
RVILQGRDSNIPGSDYINANYIKNQLLGPDENAKTYIASQGC
LEATVNDFWQMAWQENSRVIVMTTREVEKGRNKCVPYWPEVG
MQRAYGPYSVTNCGEHDTTEYKLRTLQVSPLDNGDLIREIWH
YQYLSWPDHGVPSEPGGVLSFLDQINQRQESLPHAGPIIVHC
SAGIGRTGTIIVIDMLMENISTKGLDCDIDIQKTIQMVRAQR
SGMVQTEAQYKFIYVAIAQF

Illustrative amino acid sequences for PTPN11/SHP-2 (SEQ ID NO: 19) and the PTPN11/SHP-2 phosphatase domain (SEQ ID NO: 20) are shown below.

sequence of PTPN11
SEQ ID NO: 19
MTSRRWFHPNITGVEAENLLLTRGVDGSFLARPSKSNPGDFT
LSVRRNGAVTHIKIQNTGDYYDLYGGEKFATLAELVQYYMEH
HGQLKEKNGDVIELKYPLNCADPTSERWFHGHLSGKEAEKLL
TEKGKHGSFLVRESQSHPGDFVLSVRTGDDKGESNDGKSKVT
HVMIRCQELKYDVGGGERFDSLTDLVEHYKKNPMVETLGTVL
QLKQPLNTTRINAAEIESRVRELSKLAETTDKVKQGFWEEFE
TLQQQECKLLYSRKEGQRQENKNKNRYKNILPFDHTRVVLHD
GDPNEPVSDYINANIIMPEFETKCNNSKPKKSYIATQGCLQN
TVNDFWRMVFQENSRVIVMTTKEVERGKSKCVKYWPDEYALK
EYGVMRVRNVKESAAHDYTLRELKLSKVGQALLQGNTERTVW
QYHFRTWPDHGVPSDPGGVLDFLEEVHHKQESIMDAGPVVVH
CSAGIGRTGTFIVIDILIDIIREKGVDCDIDVPKTIQMVRSQ
RSGMVQTEAQYRFIYMAVQHYIETLQRRIEEEQKSKRKGHEY
TNIKYSLADQTSGDQSPLPPCTPTPPCAEMREDSARVYENVG
LMQQQKSFR
sequence of phosphatase domain of PTPN11
SEQ ID NO: 20
FWEEFETLQQQECKLLYSRKEGQRQENKNKNRYKNILPFDHT
RVVLHDGDPNEPVSDYINANIIMPEFETKCNNSKPKKSYIAT
QGCLQNTVNDFWRMVFQENSRVIVMTTKEVERGKSKCVKYWP
DEYALKEYGVMRVRNVKESAAHDYTLRELKLSKVGQALLQGN
TERTVWQYHFRTWPDHGVPSDPGGVLDFLEEVHHKQESIMDA
GPVVVHCSAGIGRTGTFIVIDILIDIIREKGVDCDIDVPKTI
QMVRSQRSGMVQTEAQYRFIYMAVQHYIETLQRRIEEEQKSK
RKGHEYTNIKYSLADQTSGDQSPLPPCTPTPPCAEMREDSAR
VYENVGLMQQQKSFR

Further illustrative receptors which comprise an ITIM containing endodomain include, but are not limited to, CD22, LAIR-1, the Killer inhibitory receptor family (KIR), LILRB1, CTLA4, PD-1, BTLA etc. When phosphorylated, ITIMs recruits endogenous PTPN6 through its SH2 domain. If co-localised with an ITAM containing endodomain, dephosphorylation occurs and the activating signal from the CAR/TCR is reduced and/or inhibited.

The single domain protein binder may specifically bind and inhibit the activity of an inhibitory signalling molecule as described herein.

By way of example, the single domain protein binder may bind to PTPN11/SHP-2 and inhibit its dephosphorylation activity.

For example, the single domain protein binder may bind to the SH2 domain of PTPN11/SHP-2. Illustrative FN3 scaffolds that specifically bind to the SH2 domain of SHP-2 are described by Sha et al.; PNAS, 2013, vol. 110, no. 37—incorporated herein by reference. The sequences of these FN3 scaffolds are summarised in Table 4.

Modulating

The present single domain protein binder is capable of specifically binding a target protein in order to modulate activity of the target protein and, accordingly, modulate the activity of an intracellular signalling pathway in the cell of the invention.

“Modulating the activity” may refer to decreasing or increasing the activity of the target protein.

Decreasing the activity of a target protein may mean a reduction/inhibition in the activity of the target protein by at least 10%, at least 20%, at least 30%, at least 40%, at least 50%, at least 60%, at least 70%, at least 80%, at least 90%, at least 95% or at least 99%.

Increasing the activity of a target protein may mean an increase in the activity of the target protein by at least 1.2-fold, at least 1.5-fold, at least 2.0-fold, at least 2.5-fold, at least 5-fold, at least 10-fold, at least 20-fold, at least 50-fold, or at least 100-fold.

As will be apparent, increase or inhibition/reduction in activity refers to a comparison to the activity in a corresponding, control cell which is treated in identical conditions to the cell of invention, except that it does not comprise an engineered single domain protein binder as described herein.

As will be appreciated, the activity referred to will be dependent on the specific target protein; as described herein.

Reduces/Inhibit an Interaction

In one embodiment, the single domain protein binder inhibits the activity of a target protein by reducing/inhibiting an interaction between the target protein and a second entity such as a second polypeptide. As such, the protein-protein interaction of target protein and the second polypeptide may be disrupted in the presence of the single domain protein binder i.e. the single domain protein binder acts as a “disruptor” of binding between the target protein and a second polypeptide.

Suitably, binding between the target protein and a second polypeptide may be reduced by at least 10%, at least 20%, at least 30%, at least 40%, at least 50%, at least 60%, at least 70%, at least 80%, at least 90%, at least 95% or at least 99% by the single domain protein binder. Suitably, binding between the target protein and a second polypeptide may be eliminated.

The single domain protein binder may be capable of specifically binding the target protein at a higher affinity than the binding between the target protein and the second polypeptide.

As used herein, “higher affinity” means that the single domain protein binder binds to the target protein with at least 5, 10, 20, 50, 100, 1000 or 10000-fold greater affinity than the binding affinity between the target protein and the second polypeptide.

Assays for measuring binding affinity and competitive binding are known in the art such as radioactive ligand binding assays (including saturation binding, scatchard plot), non-radioactive ligand binding assays (including fluorescence polarization, fluorescence resonance energy transfer and surface plasmon resonance/Biacore, and solid phase ligand binding assays. Any method known in the art may be used to measure binding affinity of the single domain protein binder and its target protein.

Suitably, binding of the single domain protein binder may prevent the co-localisation of target protein and the second polypeptide.

Suitably, binding of the single domain protein binder may reduce or eliminate signalling downstream of target protein and the second polypeptide. Suitably, signalling downstream of target protein and the second polypeptide may be reduced by at least 10%, at least 20%, at least 30%, at least 40%, at least 50%, at least 60%, at least 70%, at least 80%, at least 90%, at least 95% or at least 99% by the single domain protein binder. Suitably, signalling downstream of target protein and the second polypeptide may be eliminated.

Reducing/inhibiting the interaction between the target protein and a second polypeptide may be achieved—for example—via competitive binding to, or steric hindrance of, the target protein.

The interaction between the target protein and the second polypeptide interaction domain may be disrupted by the single domain protein binder binding competitively to the target protein.

As used herein “competitive binding of the target protein” refers to the binding of the single domain protein binder which directly prevents binding between the target protein and the second polypeptide. An single domain protein binder may bind competitively by directly binding to binding site on the target protein which interacts with the reciprocal binding site on the second polypeptide. Alternatively, the single domain protein binder may bind competitively by binding to a region which overlaps with the binding site on the target protein which interacts with the reciprocal binding site on the second polypeptide.

Binding of the single domain protein binder to its target protein at a region that results in competitive binding of the target protein may also be referred to herein an “blocking” of the target protein.

Suitably, the single domain protein binder may reduce/inhibit an interaction between the target protein and a second polypeptide via a steric hindrance.

As used herein, “steric hindrance” refers to the single domain protein binder binding to the target protein at a site that is distinct from the binding site that facilitates the interaction between the target protein and the second polypeptide; wherein said binding between the single domain protein binder and the target protein reduces/inhibits a binding interaction between the target protein and a second polypeptide. For example, binding between the single domain protein binder and the target protein may induce a conformational shift in the target protein that reduces/inhibits binding between the target protein and the second polypeptide.

Target Degradation

In one embodiment, the single domain protein binder may sequester and/or facilitate degradation of its target protein.

Single domain protein binders (e.g. sdAbs) which are capable of directing a target protein for targeted protein degradation have been described in the art (see Caussinus et al.; Nat Struct Mol Biol; 2011; 19(1); 117-121 & Fulcher et al.; Open Biol; 2016; 6(10); pii 160255—each of which is incorporated herein by reference).

In this embodiment, the single domain protein binder may further comprise a degradation motif which targets the target protein for e.g. proteasome-mediated protein degradation.

The predominant proteasome in mammals is the cytosolic 26S proteasome, which is about 2000 kDa and contains one 20S protein subunit and two 19S regulatory cap subunits. The core is hollow and provides an enclosed cavity in which proteins are degraded; openings at the two ends of the core allow the target protein to enter. Each end of the core particle associates with a 19S regulatory subunit that contains multiple ATPase active sites and ubiquitin binding sites; it is this structure that recognizes polyubiquitinated proteins and transfers them to the catalytic core.

Proteins are targeted for degradation by the proteasome with covalent modification of a lysine residue that requires the coordinated reactions of three enzymes. In the first step, a ubiquitin-activating enzyme (known as E1) hydrolyzes ATP and adenylylates a ubiquitin molecule. This is then transferred to E1's active-site cysteine residue in concert with the adenylylation of a second ubiquitin. This adenylylated ubiquitin is then transferred to a cysteine of a second enzyme, ubiquitin-conjugating enzyme (E2). In the last step, a member of a highly diverse class of enzymes known as ubiquitin ligases (E3) recognizes the specific protein to be ubiquitinated and catalyzes the transfer of ubiquitin from E2 to this target protein. A target protein must be labeled with at least four ubiquitin monomers (in the form of a polyubiquitin chain) before it is recognized by the proteasome lid. It is therefore the E3 that confers substrate specificity to this system.

Accordingly, a “degradation motif” as used herein may refer to an E3 domain which catalyses the transfer of ubiquitin from E2 to the target protein. Herein, recognition of the target protein is mediated by a binding domain in the single domain protein binder (e.g. a sdAb or non-antibody scaffold as described herein). Once the ubiquitin is transferred, the target protein is subject to proteasome-mediated degradation.

Thus, in this embodiment the single domain protein binder reduces the level of target protein in the cell—thereby reducing/inhibiting the activity of the target protein and the intracellular signalling pathway in which it functions.

Suitable degradation motifs are known in the art. Such degradation motifs include, but are not limited to, the amino acid sequences shown as SEQ ID NO: 21, 22, 30, 31 or 32.

(E3 ubiquitin-protein ligase CHIP)
SEQ ID NO: 21
RLNFGDDIPSALRIAKKKRWNSIEERRIHQE
SELHSYLSRLIAAERERELEECQRNHEGDED
DSHVRAQQACIEAKHDKYMADMDELFSQVDE
KRKKRDIPDYLCGKISFELMREPCITPSGIT
YDRKDIEEHLQRVGHFDPVIRSPLIQEQLIP
NLAMKEVIDAFISENGWV
(Simb)
SEQ ID NO: 22
MMKMETDKIMDETNSNAQAFTTTMLYDPVRK
KDSSPTYQTERELCFQYFTQWSESGQVDFVE
HLLSRMCHYQHGQINAYLKPMLQRDFITLLP
IKGLDHIAENILSYLDAESLKSSELVCKEWL
RVISEGMLWKKLIERKVRTDSLWRGLAERRN
WMQYLFKPRPGQTQRPHSFHRELFPKIMNDI
DSIENNWRTGRH
(XIAP's RING domain)
SEQ ID NO: 30
CKICMDRNIAIVFVPCGHLVTCKQCAEAVDK
CPMCYTVITFKQKIFMS
(e4B's U-box)
SEQ ID No: 31
DAPDEFRDPLMDTLMTDPVRLPSGTIMDRSI
ILRHLLNSPTDPFNRQTLTESMLEPVPELKE
QIQAWMREKQNSDH
(Notch1's PEST motif)
SEQ ID No. 32
HPFLTPSPESPDQWSSSSPHSNVSDWSEGVS
SPPTSMQSQIARIPEAFK

Suitably, the degradation motif may comprise a sequence shown as SEQ ID NO: 21, 22, 30, 31 or 32. or a variant thereof. Variant sequences may have at least 80%, 85%, 90%, 95%, 98% or 99% sequence identity provided that the variant is capable of catalysing the transfer of ubiquitin from E2 to the target protein.

In another embodiment, the single domain protein binder may comprise a motif which causes it to be directed for protein degradation (e.g. proteasome-mediated degradation). As such, binding of the single domain protein binder to the target protein will result in target protein becoming target for protein degradation.

Suitable degradation motifs are known in the art and include, but are not limited to, the PEST domain. PEST domains are rich in proline (P), glutamic acid (E), serine (S), and threonine (T). These PEST domains are typically flanked by clusters containing several positively charged amino acids.

An illustrative PEST domain sequence is shown as SEQ ID NO: 23. Suitably, the PEST domain may comprise a sequence shown as SEQ ID NO: 23 or a variant thereof.

Variant sequences of SEQ ID NO: 23 may have at least 80%, 85%, 90%, 95%, 98% or 99% sequence identity to SEQ ID NO: 23; provided that the variant is capable of mediating degradation of the target protein.

(PEST)
SEQ ID NO: 23
HGFPPEVEEQDDGTLPMSCAQESGMDRHPAA
CASARINV

Suitable assays for determining whether a target protein has been subject to degradation are known in the art and include, for example, the assays described in Caussinus et al. (Nat. Struct. Amp Mol. Biol. 19, 117 (2011), incorporated herein by reference).

Sequestration

In one embodiment, the single domain protein binder may sequester its target protein. For example, the single domain protein binder may have a KDEL-like motif at the extreme C terminus. Polypeptides with a KDEL motif are sorted and retrieved back to the ER from the intermediate compartment of cis-Golgi during the early stages of the secretory pathway. Single domain protein binders (e.g. sdAbs) which contain KDEL-like motif are capable of sequestering a target protein within the ER. In addition, polypeptides may be targeted to other destinations, such as mitochondria or the nucleus away from subcellular locations where interacting polypeptides reside.

Activation

As described herein, the single domain protein binder may cause activation of a target protein. For example, the single domain protein binder may cause activation of a target protein by mediating dimerization, trimerization or oligomerization of a target protein which is required for activation.

Examples of such mechanisms for mediating activation are described herein.

Tuning

Suitably, expression of the single domain protein binder as described herein may be “tunable”.

As used herein, “tunable” means that it is possible to increase, decrease, turn on or turn off expression of the engineered intracellular binding protein by the engineered immune effector cell.

Suitably, expression of the single domain protein binder may be controlled or tuned by an inducible promoter. Suitably, the secreted factor may be regulated by Nuclear factor of activated T cells (NFAT) response element.

An NFAT response element may comprise the nucleotide sequence set forth in SEQ ID NO: 24 or a variant thereof.

(SEQ ID NO: 24)
GGAGGAAAAACTGTTTCATACAGAAGGCGT.

Variant sequences of SEQ ID NO: 24 may have at least 80%, 85%, 90%, 95%, 98% or 99% sequence identity to SEQ ID NO: 24. Suitably, the variant sequence is able to function as a NFAT response element.

The NFAT response element may comprise repeat units such as 3, 4, 5 or 6 repeat units. Suitably, the NFAT response element may comprise 3, 4, 5 or 6 repeat units of SEQ ID NO: 24. The NFAT response element may be positioned in front of a promoter (e.g. a CMV promoter).

Cell

The present invention relates to a cell. In particular, the present invention relates to an engineered immune cell.

An “engineered cell” as used herein means a cell which has been modified to comprise or express a nucleic acid sequence which is not naturally encoded by the cell. Methods for engineering cells are known in the art and include but are not limited to genetic modification of cells e.g. by transduction such as retroviral or lentiviral transduction, transfection (such as transient transfection—DNA or RNA based) including lipofection, polyethylene glycol, calcium phosphate and electroporation. Any suitable method may be used to introduce a nucleic acid sequence into a cell.

Accordingly, the nucleic acid sequences encoding the engineered single domain protein binder which is capable of modulating an intracellular signalling pathway; and CAR or transgenic TCR respectively are not naturally expressed by a corresponding, unmodified cell—for example an unmodified alpha-beta T cell, a NK cell, a gamma-delta T cell or cytokine-induced killer cell.

Suitably, an engineered cell is a cell whose genome has been modified e.g. by transduction or by transfection. Suitably, an engineered cell is a cell whose genome has been modified by retroviral transduction. Suitably, an engineered cell is a cell whose genome has been modified by lentiviral transduction.

As used herein, the term “introduced” refers to methods for inserting foreign DNA or RNA into a cell. As used herein the term introduced includes both transduction and transfection methods. Transfection is the process of introducing nucleic acids into a cell by non-viral methods. Transduction is the process of introducing foreign DNA or RNA into a cell via a viral vector.

Engineered cells according to the present invention may be generated by introducing DNA or RNA coding for the releasable protein and the retention protein by one of many means including transduction with a viral vector, transfection with DNA or RNA.

Cells may be activated and/or expanded prior to the introduction of a nucleic acid sequence(s) encoding the engineered single domain protein binder and a CAR or transgenic TCR, for example by treatment with an anti-CD3 monoclonal antibody or both anti-CD3 and anti-CD28 monoclonal antibodies. As used herein “activated” means that a cell has been stimulated, causing the cell to proliferate, differentiate or initiate an effector function.

Methods for measuring cell activation are known in the art and include, for example, measuring the expression of activation markers by flow cytometry, such as the expression of CD69, CD25, CD38 or HLA-DR or measuring intracellular cytokines.

As used herein “expanded” means that a cell or population of cells has been induced to proliferate.

The expansion of a population of cells may be measured for example by counting the number of cells present in a population. The phenotype of the cells may be determined by methods known in the art such as flow cytometry.

“Cytolytic immune cell” as used herein is a cell which directly kills other cells. Cytolytic cells may kill cancerous cells; virally infected cells or other damaged cells. Cytolytic immune cells include T cells and Natural killer (NK) cells.

Cytolytic immune cells can be T cells or T lymphocytes which are a type of lymphocyte that play a central role in cell-mediated immunity. T cells can be distinguished from other lymphocytes, such as B cells and NK cells, by the presence of a TCR on their cell surface.

Cytolytic T cells (TC cells, or CTLs) destroy virally infected cells and tumour cells, and are also implicated in transplant rejection. CTLs express the CD8 at their surface. CTLs may be known as CD8+ T cells. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Suitably, the cell of the present invention may be a T-cell. Suitably, the T cell may be an alpha-beta T cell. Suitably, the T cell may be a gamma-delta T cell.

Natural Killer Cells (or NK cells) are a type of cytolytic cell which form part of the innate immune system. NK cells provide rapid responses to innate signals from virally infected cells in an MHC independent manner.

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

Suitably, the cell of the present invention may be a wild-type killer (NK) cell. Suitably, the cell of the present invention may be a cytokine induced killer cell.

The cell may be derived from a patient's own peripheral blood (1st party), or in the setting of a haematopoietic stem cell transplant from donor peripheral blood (2nd party), or peripheral blood from an unconnected donor (3rd party). T or NK cells, for example, may be activated and/or expanded prior to being transduced with nucleic acid molecule(s) encoding the polypeptides of the invention, for example by treatment with an anti-CD3 monoclonal antibody.

Alternatively, the cell may be derived from ex vivo differentiation of inducible progenitor cells or embryonic progenitor cells to T cells. Alternatively, an immortalized T-cell line which retains its lytic function may be used.

The cell may be a haematopoietic stem cell (HSC). HSCs can be obtained for transplant from the bone marrow of a suitably matched donor, by leukapheresis of peripheral blood after mobilization by administration of pharmacological doses of cytokines such as G-CSF [peripheral blood stem cells (PBSCs)], or from the umbilical cord blood (UCB) collected from the placenta after delivery. The marrow, PBSCs, or UCB may be transplanted without processing, or the HSCs may be enriched by immune selection with a monoclonal antibody to the CD34 surface antigen.

Chimeric Antigen Receptor

Classical CARs are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site or on a ligand for the target antigen. A spacer domain may be necessary to isolate the binder from the membrane and to allow it a suitable orientation. A common spacer domain used is the Fc of IgG1. More compact spacers can suffice e.g. the stalk from CD8a and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FccR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 4-1BB which transmit survival signals. Even more potent third generation CARs have now been described which have endodomains capable of transmitting activation, proliferation and survival signals.

CAR-encoding nucleic acids may be transferred to T cells using, for example, retroviral vectors. In this way, a large number of antigen-specific T cells can be generated for adoptive cell transfer. When the CAR binds the target-antigen, this results in the transmission of an activating signal to the T-cell it is expressed on. Thus the CAR directs the specificity and cytotoxicity of the T cell towards cells expressing the targeted antigen.

Antigen Binding Domain

The antigen-binding domain is the portion of a classical CAR which recognizes antigen.

Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a wild-type ligand of the target antigen; a peptide with sufficient affinity for the target; a single domain binder such as a camelid; an artificial binder single as a Darpin; or a single-chain derived from a T-cell receptor.

Various tumour associated antigens (TAA) are known, as shown in the following Table 1. The antigen-binding domain used in the present invention may be a domain which is capable of binding a TAA as indicated therein.

TABLE 1

| Cancer type | TAA |
|---|---|
| Diffuse Large B-cell Lymphoma | CD19, CD20 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

Transmembrane Domain

The transmembrane domain is the sequence of a classical CAR that spans the membrane. It may comprise a hydrophobic alpha helix. The transmembrane domain may be derived from CD28, which gives good receptor stability.

CAR or TCR Signal Peptide

The CAR or transgenic TCR for use in the present invention may comprise a signal peptide so that when it is expressed in a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

Spacer Domain

The receptor may comprise a spacer sequence to connect the antigen-binding domain with the transmembrane domain. A flexible spacer allows the antigen-binding domain to orient in different directions to facilitate binding.

The spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

Intracellular Signalling Domain

The intracellular signalling domain is the signal-transmission portion of a classical CAR.

The most commonly used signalling domain component is that of CD3-zeta endodomain, which contains 3 ITAMs. This transmits an activation signal to the T cell after antigen is bound. CD3-zeta may not provide a fully competent activation signal and additional co-stimulatory signalling may be needed. For example, chimeric CD28 and OX40 can be used with CD3-Zeta to transmit a proliferative/survival signal, or all three can be used together.

The intracellular signalling domain may be or comprise a T cell signalling domain.

The intracellular signalling domain may comprise one or more immunoreceptor tyrosine-based activation motifs (ITAMs). An ITAM is a conserved sequence of four amino acids that is repeated twice in the cytoplasmic tails of certain cell surface proteins of the immune system. The motif contains a tyrosine separated from a leucine or isoleucine by any two other amino acids, giving the signature YxxL/I. Two of these signatures are typically separated by between 6 and 8 amino acids in the tail of the molecule (YxxL/Ix₍₆₋₈₎YxxL/I).

ITAMs are important for signal transduction in immune cells. Hence, they are found in the tails of important cell signalling molecules such as the CD3 and ζ-chains of the T cell receptor complex, the CD79 alpha and beta chains of the B cell receptor complex, and certain Fc receptors. The tyrosine residues within these motifs become phosphorylated following interaction of the receptor molecules with their ligands and form docking sites for other proteins involved in the signalling pathways of the cell.

The intracellular signalling domain component may comprise, consist essentially of, or consist of the CD3-ζ endodomain, which contains three ITAMs. Classically, the CD3-ζ endodomain transmits an activation signal to the T cell after antigen is bound.

The intracellular signalling domain may comprise additional co-stimulatory signalling. For example, 4-1 BB (also known as CD137) can be used with CD3-ζ, or CD28 and OX40 can be used with CD3-ζ to transmit a proliferative/survival signal.

Suitably, the CAR may have the general format: antigen-binding domain-TCR element.

As used herein “TCR element” means a domain or portion thereof of a component of the TCR receptor complex. The TCR element may comprise (e.g. have) an extracellular domain and/or a transmembrane domain and/or an intracellular domain e.g. intracellular signalling domain of a TCR element.

The TCR element may selected from TCR alpha chain, TCR beta chain, a CD3 epsilon chain, a CD3 gamma chain, a CD3 delta chain, CD3 epsilon chain.

Suitably, the TCR element may comprise the extracellular domain of the TCR alpha chain, TCR beta chain, a CD3 epsilon chain, a CD3 gamma chain, a CD3 delta chain, or CD3 epsilon chain. Suitably, the TCR element may comprise the transmembrane domain of the TCR alpha chain, TCR beta chain, a CD3 epsilon chain, a CD3 gamma chain, a CD3 delta chain, or CD3 epsilon chain. Suitably, the TCR element may comprise the intracellular domain of the TCR alpha chain, TCR beta chain, a CD3 epsilon chain, a CD3 gamma chain, a CD3 delta chain, or CD3 epsilon chain. Suitably, the TCR element may comprise the TCR alpha chain, TCR beta chain, a CD3 epsilon chain, a CD3 gamma chain, a CD3 delta chain, or CD3 epsilon chain.

Transgenic T-Cell Receptor (TCR)

The T-cell receptor (TCR) is a molecule found on the surface of T cells which is responsible for recognizing fragments of antigen as peptides bound to major histocompatibility complex (MHC) molecules.

The TCR is a heterodimer composed of two different protein chains. In humans, in 95% of T cells the TCR consists of an alpha (α) chain and a beta (β) chain (encoded by TRA and TRB, respectively), whereas in 5% of T cells the TCR consists of gamma and delta (γ/δ) chains (encoded by TRG and TRD, respectively).

When the TCR engages with antigenic peptide and MHC (peptide/MHC), the T lymphocyte is activated through signal transduction.

In contrast to conventional antibody-directed target antigens, antigens recognized by the TCR can include the entire array of potential intracellular proteins, which are processed and delivered to the cell surface as a peptide/MHC complex.

It is possible to engineer cells to express heterologous (i.e. non-native) TCR molecules by artificially introducing the TRA and TRB genes; or TRG and TRD genes into the cell using a vector. For example the genes for engineered TCRs may be reintroduced into autologous T cells and transferred back into patients for T cell adoptive therapies. Such ‘heterologous’ TCRs may also be referred to herein as ‘transgenic TCRs’.

Nucleic Acid Construct

The present invention provides a nucleic acid construct which comprises (i) a first nucleic acid sequence which encodes a single domain protein binder which is capable of modulating an intracellular signalling pathway; and (ii) a second nucleic acid sequence which encodes a chimeric antigen receptor (CAR) or a transgenic T cell receptor (TCR).

The present invention also provides a kit comprising nucleic acid sequences according to the present invention. For example, the kit may comprise (i) a first nucleic acid sequence which encodes a single domain protein binder which is capable of modulating an intracellular signalling pathway; and (ii) a second nucleic acid sequence which encodes a chimeric antigen receptor (CAR) or a transgenic T cell receptor (TCR).

As used herein, the terms “polynucleotide”, “nucleotide”, and “nucleic acid” are intended to be synonymous with each other.

Suitably, the nucleic acid construct may comprise a plurality of nucleic acid sequences which encode components of the invention such as an engineered single domain protein binder and/or a CAR or transgenic TCR provided by the present invention. For example, the nucleic acid construct may comprise two, three, four or more nucleic acid sequences which encode different components of the invention. Suitably, the plurality of nucleic acid sequences may be separated by co-expression sites as defined herein.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described herein to reflect the codon usage of any particular host organism in which the polypeptides are to be expressed. Suitably, the polynucleotides of the present invention are codon optimised to enable expression in a mammalian cell, in particular a cell as described herein.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art.

Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

Co-Expression Site

A co-expression site is used herein to refer to a nucleic acid sequence enabling co-expression of nucleic acid sequences encoding the releasable protein and the retention protein of the present invention.

The co-expression site may be a sequence encoding a cleavage site, such that the engineered polynucleotide encodes the enzymes of the transgenic synthetic biology pathway joined by a cleavage site(s). Typically, a co-expression site is located between adjacent polynucleotide sequences which encode separate enzymes of the transgenic synthetic biology pathway.

Suitably, in embodiments where a plurality of co-expression sites are present in the engineered polynucleotide, the same co-expression site may be used.

Preferably, the co-expression site is a cleavage site. The cleavage site may be any sequence which enables the two polypeptides to become separated. The cleavage site may be self-cleaving, such that when the polypeptide is produced, it is immediately cleaved into individual peptides without the need for any external cleavage activity.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may be a furin cleavage site. Furin is an enzyme which belongs to the subtilisin-like proprotein convertase family. The members of this family are proprotein convertases that process latent precursor proteins into their biologically active products. Furin is a calcium-dependent serine endoprotease that can efficiently cleave precursor proteins at their paired basic amino acid processing sites. Examples of furin substrates include proparathyroid hormone, transforming growth factor beta 1 precursor, proalbumin, pro-beta-secretase, membrane type-1 matrix metalloproteinase, beta subunit of pro-nerve growth factor and von Willebrand factor. Furin cleaves proteins just downstream of a basic amino acid target sequence (canonically, Arg-X-(Arg/Lys)-Arg′) and is enriched in the Golgi apparatus.

The cleavage site may be a Tobacco Etch Virus (TEV) cleavage site.

TEV protease is a highly sequence-specific cysteine protease which is chymotrypsin-like proteases. It is very specific for its target cleavage site and is therefore frequently used for the controlled cleavage of fusion proteins both in vitro and in vivo. The consensus TEV cleavage site is ENLYFQ\S (where ‘\’ denotes the cleaved peptide bond). Mammalian cells, such as human cells, do not express TEV protease. Thus in embodiments in which the present nucleic acid construct comprises a TEV cleavage site and is expressed in a mammalian cell—exogenous TEV protease must also expressed in the mammalian cell.

The cleavage site may encode a self-cleaving peptide. A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al., 2001) as above.

The co-expression sequence may be an internal ribosome entry sequence (IRES). The co-expressing sequence may be an internal promoter.

Vector

The present invention also provides a vector, or kit of vectors which comprises one or more nucleic acid sequence(s) or nucleic acid construct(s) of the invention. Such a vector may be used to introduce the nucleic acid sequence(s) or construct(s) into a host cell so that it expresses an engineered single domain protein binder and a CAR or transgenic TCR as defined herein.

Suitably, the vector may comprise a plurality of nucleic acid sequences which encode different components as provided by the present invention. For example, the vector may comprise two, three, four or more nucleic acid sequences which encode different polypeptides of the invention, such as the engineered single domain protein binder and a CAR or transgenic TCR. Suitably, the plurality of nucleic acid sequences may be separated by co-expression sites as defined herein.

The vector may, for example, be a plasmid or a viral vector, such as a retroviral vector or a lentiviral vector, or a transposon based vector or synthetic mRNA.

The vector may be capable of transfecting or transducing a cell.

Pharmaceutical Composition

The present invention also relates to a pharmaceutical composition comprising an engineered cell according to the present invention or a cell obtainable (e.g. obtained) by a method according to the present invention.

The present invention also provides a pharmaceutical composition comprising, a nucleic acid construct according to the present invention, a first, second and third nucleic acid sequence as defined herein; a vector according to the present invention or a first, second and third vector as described herein. In particular, the invention relates to a pharmaceutical composition containing a cell according to the present invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method

The present invention provides a method for treating and/or preventing a disease which comprises the step of administering a cell according to the invention, or obtainable (e.g. obtained) by a method according to the present invention, or a nucleic acid construct according to the present invention, or a first, second and third nucleic acid sequence as defined herein; a vector according to the present invention or a first, second and third vector as described herein (for example in a pharmaceutical composition as described above) to a subject.

Suitably, the present methods for treating and/or preventing a disease may comprise administering a cell according to the present invention (for example in a pharmaceutical composition as described above) to a subject.

A method for treating a disease relates to the therapeutic use of the cells of the present invention. In this respect, the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. In this respect, the cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:

(i) isolating a cell-containing sample;
(ii) introducing the nucleic acid construct according to the present invention, a first, second and third nucleic acid sequence as defined herein, a vector according to the present invention or a first, second and third vector as herein to the cell; and
(iii) administering the cells from (ii) to a subject.

Suitably, the nucleic acid construct, vector(s) or nucleic acids may be introduced by transduction. Suitably, the nucleic acid construct, vector(s) or nucleic acids may be introduced by transfection.

Suitably, the cell may be autologous. Suitably, the cell may be allogenic.

The methods provided by the present invention for treating a disease may involve monitoring the progression of the disease and/or any toxic activity.

“Monitoring the progression of the disease” means to assess the symptoms associated with the disease over time to determine if they are reducing/improving or increasing/worsening.

“Toxic activity” relates to adverse effects caused by the cells of the invention following their administration to a subject. Toxic activities may include, for example, immunological toxicity, biliary toxicity and respiratory distress syndrome.

The present invention provides a cell according to the present invention, a nucleic acid construct according to the present invention, a first, second and third nucleic acid sequence as defined herein, a vector according to the present invention, or a first, second and third vector according to the present invention for use in treating and/or preventing a disease. In particular the present invention provides a cell of the present invention for use in treating and/or preventing a disease.

The present invention also relates to a cell according to the present invention, a nucleic acid construct according to the present invention, a first, second and third nucleic acid sequence as defined herein, a vector according to the present invention, or a first, second and third vector according to the present invention in the manufacture of a medicament for the treatment and/or prevention of a disease. In particular, the invention relates to the use of a cell according to the present invention in the manufacture of a medicament for the treatment and/or prevention of a disease.

The disease to be treated and/or prevented by the method of the present invention may be cancer.

Suitably, the cancer may be a solid tumour.

The cancer may be a cancer such as neuroblastoma, prostate cancer, bladder cancer, breast cancer, colon cancer, endometrial cancer, kidney cancer (renal cell), leukaemia, lung cancer, melanoma, non-Hodgkin lymphoma, pancreatic cancer, and thyroid cancer.

The cell of the present invention may be capable of killing target cells, such as cancer cells. The target cell may be recognisable by expression of a TAA, for example the expression of a TAA provided above in Table 1. The cancer may be a cancer listed in Table 1.

Method of Making a Cell

A cell of the present invention may be generated by introducing DNA or RNA coding for the POI, protein interaction domains and intracellular retention domain as defined herein by one of many means including transduction with a viral vector, transfection with DNA or RNA.

The cell of the invention may be made by introducing to a cell (e.g. by transduction or transfection) the nucleic acid construct or vector according to the present invention, or a first, second and third nucleic acid sequence as defined above, or a first, second and third vector as defined above.

Suitably, the cell may be from a sample isolated from a subject.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1

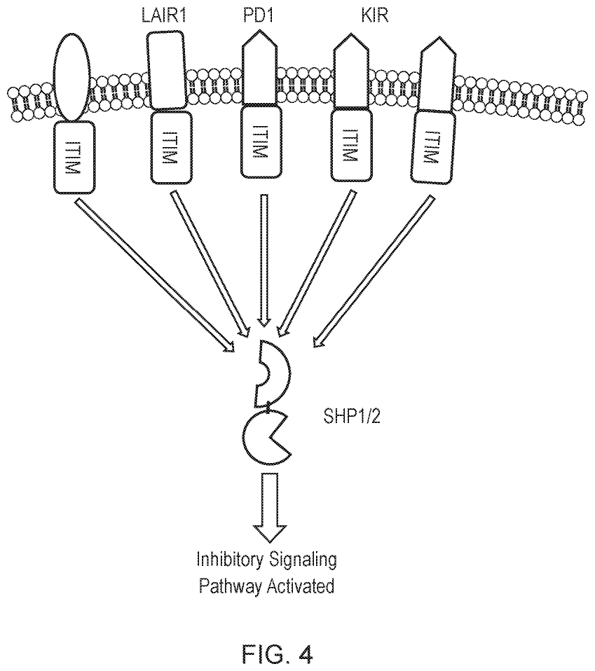

T cells are transduced to express a chimeric antigen receptor (CAR), an inhibitory receptor PD1 and a blocking intrabody to SHP2 (FIG. 4) are co-cultured for 72 hours with SupT1 target cells transduced to express the cognate ligand for the CAR and the inhibitory ligand PDL1. The ability of the T-cells to lyse targets and proliferate are measured by flow cytometry and the release of IFNy is measured by ELISA.

### Example 2

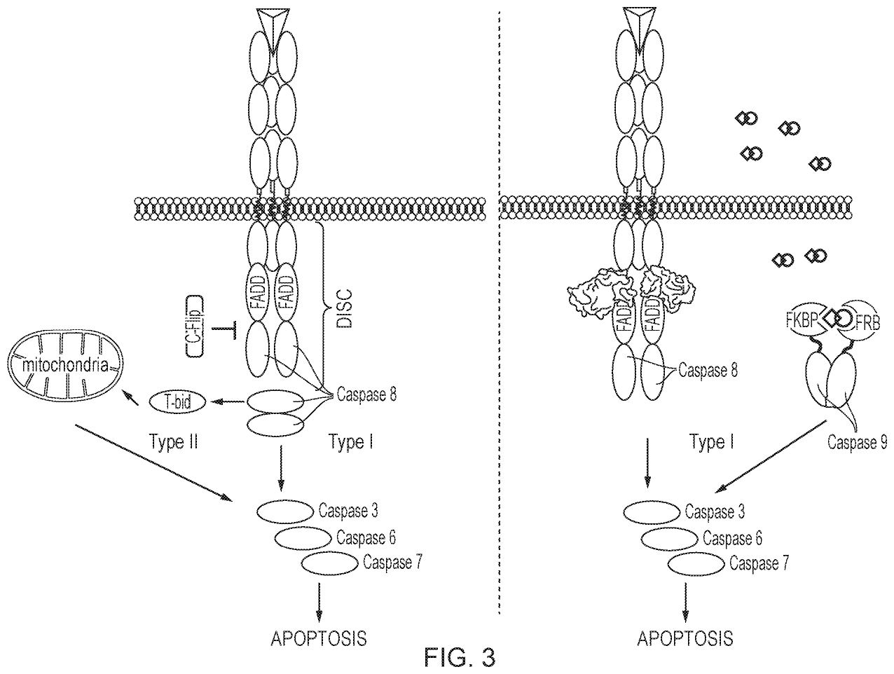

T cells are transduced to express FAS, a blocking intrabody to FADD and a marker of transduction eGFP (FIG. 3). FAS induced apoptosis is measured after 24 h exposure to soluble FASL or crosslinking antibodies to FAS. The number of GFP positive T cells that have undergone apoptosis is measured by 7AAD and annexin V stain on flow cytometry. Molecular interactions are tested through phospho-serine western blot on cell lysate directed to S194 on FADD and the presence of cleaved Caspases.

### Example 3

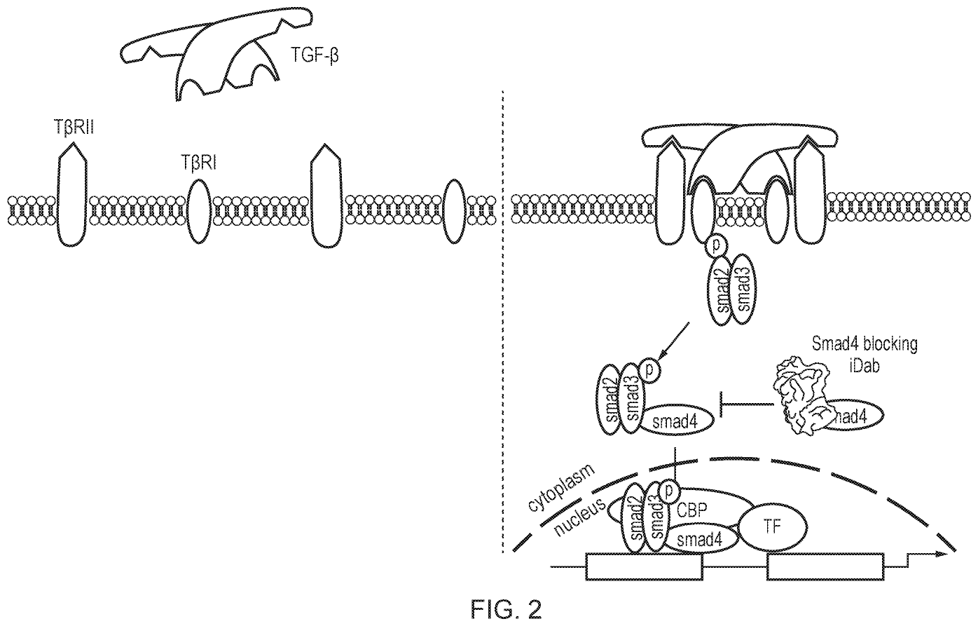

T cells are transduced to express a chimeric antigen receptor (CAR) and a blocking intrabody to SMAD4 (FIG. 2) are co-cultured for 72 hours with SupT1 target cells transduced to express the cognate ligand for the CAR and soluble TGFbeta1. The ability of the T-cells to lyse targets and proliferate are measured by flow cytometry and the release of IFNy is measured by ELISA. Molecular interactions are tested by immunoprecipitation of cell lysate for SMAD4 followed by a western blot of SMAD2/3 (FIG. 3).

### Example 4

T cells are transduced to express FAS, FLIP_{L} and a marker of transduction eGFP. FAS induced apoptosis is measured after 24 h exposure to soluble FASL or crosslinking antibodies to FAS. The number of GFP positive T cells that have undergone apoptosis is measured by 7AAD and annexin V stain on flow cytometry. Molecular interactions are tested through phospho-serine western blot on cell lysate directed to S194 on FADD and the presence of cleaved Caspases.

### Example 5—FN3 Based Binding Domains Targeting SHP2

FN3 based monobodies were engineered by phage display to SHP2 and a selection of binders were isolated, some of which targeted the C-terminal SH2 domain and some of which targeted the N-terminal domain of SHP2. The binders are described in Sha et al. 2013 (PNAS vol. 110, no. 37: 14924-14929) and their amino acid sequences are shown in Table 4 above.

Binding of the FN3 based monobodies to the N or C terminal SH2 domain of SHP2 was investigated by ELISA and specific binding to the reported epitopes was confirmed (data not shown).

A panel of effector T cells (Jurkats) were created expressing the GD2 CAR described in WO2015/132604, PD1 and/or an FN3 based monobody (Nsa1 or Nsa5) as shown below:

| Name | Description |
|---|---|
| Jurkats NT | Non-transduced cells (negative control) |
| CAR+ | Transduced with construct expressing |
| | GD2-CAR only |
| PD1 + CAR+ | Transduced with construct expressing |
| | PD1 and GD2 CAR |
| Nterm1 + PD1 + CAR | Transduced with construct expressing Nsa1 |
| | monobody, PD1 and GD2 CAR |
| Nterm4 + PD1 + CAR | Transduced with construct expressing Nsa5 |
| | monobody, PD1 and GD2 CAR |

SupT1 target cells were created expressing GD2 optionally in combination with PD-L1, as follows:

| Name | Description |
|---|---|
| Supt1 NT | Non-transduced cells (negative control) |
| GD2+ | Transduced with construct expressing GD2 target antigen |
| GD2 + PD1I | Transduced with construct expressing GD2 target antigen |
| | and PD-L1 |

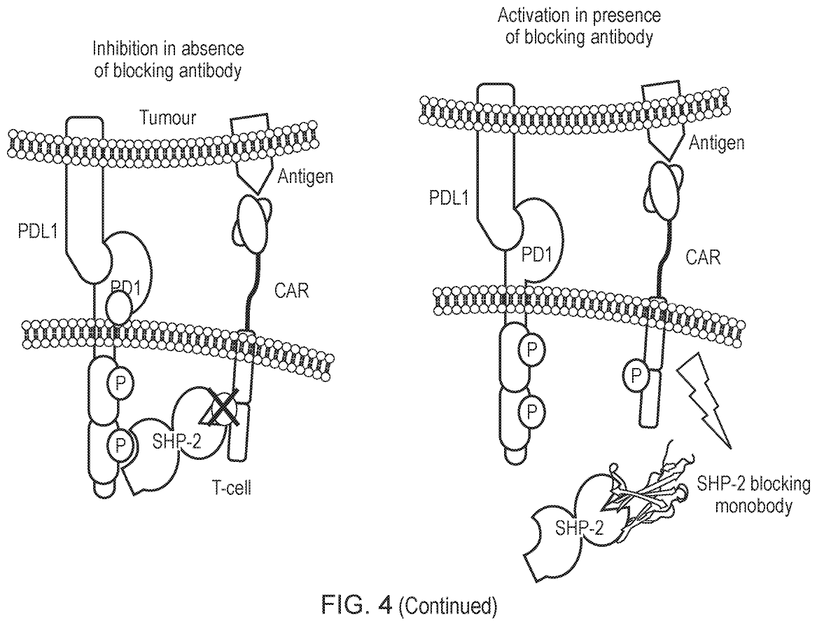

Cells were co-cultured for 24 hours and CD69 expression analysed by FACS. The results are shown in FIG. 5. T cell activation is inhibited in the presence of PD1/PD-L1. This inhibition is partially alleviated by co-expression of either the Nsa1 or Nsa5 FN3 based binding domain targeting SHP2.

### Example 6—Fusing Intra-dAbs to Degradation Motifs

A series of constructs were designed in order to investigate the effect of linking an intra-dAb to various protein interaction domains which are found in the junctions of the ubiquitin relay system.

The general design of the constructs was as follows:
RQR8-2A-aBFP VHH-L-protein interaction domain

in which:

“RQR8” is a marker gene;

“2A” is an FMDV derived peptide enabling co-expression of the marker, together with the intra-dAb+degradadtion motif;

“aBFP VHH” is a domain antibody against the fluorescent protein BFP

“L” is a linker

“protein interaction domain” is a domain with E3 ligase activity, selected from: XIAP's RING domain (SEQ ID No. 30); e4B's U-box (SEQ ID No. 31) and Notch1's PEST motif (SEQ ID No. 2).

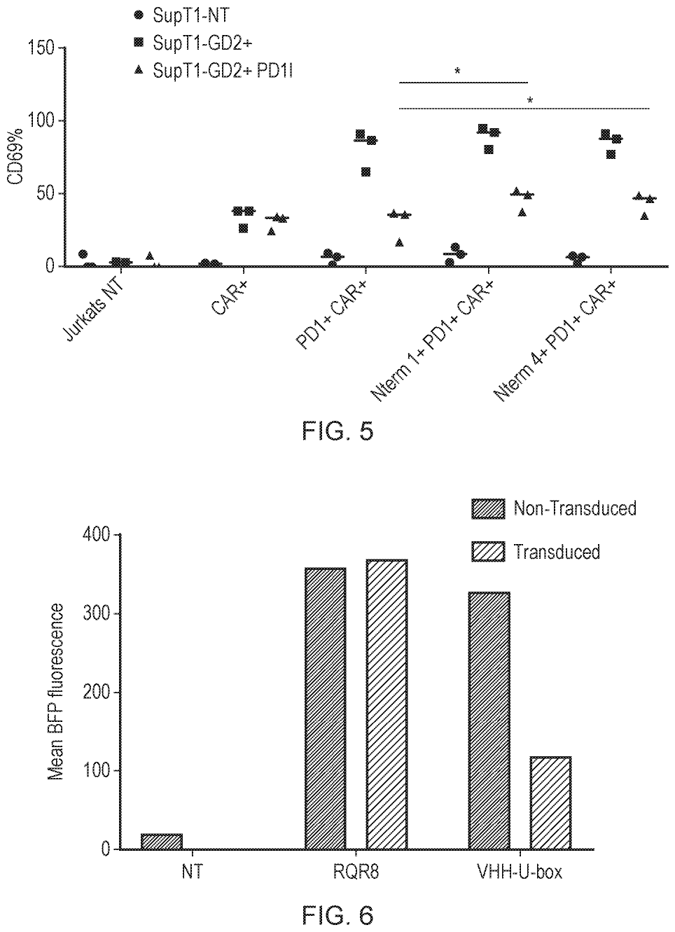

Raji cells expressing BFP were transduced with either the RQR8 marker gene alone (control) or with one of the constructs above. The results for aBFP VHH linked to e4B's U-box are shown in FIG. 6. Transduction with the intra-dAb linked to Ubox caused a reduction in fluorescence, indicating that BFP within the cell is being degraded.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A cell which expresses:
(i) an intracellular single domain antibody or non-antibody scaffold which binds a target protein and modulates an intracellular signalling pathway; and
(ii) a chimeric antigen receptor (CAR) or a transgenic T cell receptor (TCR).

2-5. (canceled)

6. A cell according to claim 1 wherein the single domain antibody or non-antibody scaffold:
(i) reduces or prevents an interaction between its target protein and a second polypeptide;
(ii) sequesters and/or facilitates degradation of its target protein; or
(iii) activates its target protein.

7. A cell according to claim 6 wherein the single domain antibody or non-antibody scaffold reduces or prevents an interaction between its target protein and a second polypeptide by blocking or sterically hindering the binding site of the target protein for the second polypeptide.

8. A cell according to claim 6 wherein the single domain antibody or non-antibody scaffold comprises a degradation motif and targets the target protein for proteasome-mediated degradation.

9-11. (canceled)

12. A cell according to claim 11, wherein the single domain antibody or non-antibody scaffold binds or blocks SHP-2.

13-14. (canceled)

15. A cell according to claim 10 wherein the single domain antibody or non-antibody scaffold inhibits the activity of a proapoptotic signalling polypeptide.

16. A cell according to claim 15 wherein the proapoptotic signalling polypeptide is selected from FAS, FADD, P38, P53, CARD protein, BCL10, Capase 9, and Apaf1.

17. A cell according to claim 16 wherein the single domain antibody or non-antibody scaffold is (i) a FAS or FADD blocking polypeptide; or (ii) targets FAS or FADD for proteasome-mediated degradation.

18. A cell according to claim 17 wherein the single domain antibody or non-antibody scaffold binds the FAS cytoplasmic domain or the FADD Death domain.

19. A cell according to claim 10 wherein the intracellular signalling pathway is the TGFβ pathway.

20. A cell according to claim 19, wherein the single domain antibody or non-antibody scaffold protein binder reduces or prevents a binding interaction between SMAD4 and the SMAD3/2 complex.

21. A cell according to claim 19 wherein the single domain antibody or non-antibody scaffold is a SMAD4 blocking polypeptide.

22. A cell according to claim 21, wherein the single domain antibody or non-antibody scaffold binds and blocks the SMAD4 MH2 domain.

23. (canceled)

24. A nucleic acid construct which comprises: (i) a first nucleic acid sequence which encodes a single domain antibody or non-antibody scaffold which binds a target protein and modulates an intracellular signaling pathway; and (ii) a second nucleic acid sequence which encodes a chimeric antigen receptor (CAR) or a transgenic T cell receptor (TCR).

25-26. (canceled)

27. A vector which comprises a nucleic acid construct according to claim 24.

28. A kit of vectors which comprises:
(i) a first vector which comprises a nucleic acid sequence which encodes a single domain antibody or non-antibody scaffold which binds a target protein and modulates an intracellular signaling pathway; and
(ii) a second vector which comprises a nucleic acid sequence which encodes a chimeric antigen receptor (CAR) or a transgenic T cell receptor (TCR).

29. A pharmaceutical composition which comprises a plurality of cells according to claim 1.

30. (canceled)

31. A method for treating and/or preventing a disease, which comprises the step of administering a pharmaceutical composition according to claim 29 to a subject in need thereof.

32-34. (canceled)

35. A method for making a cell according to claim 1, which comprises the step of introducing into a cell a nucleic acid sequence encoding an antibody or non-antibody scaffold which binds a target protein and modulates an intracellular signaling pathway.

36. (canceled)
